(19) United States
(12) Patent Application Publication
(10) Pub. No.: US 2011/0288159 A1
(43) Pub. Date: Nov. 24, 2011

(54) METHODS OF EXPRESSING A POLYPEPTIDE IN THE BRAIN AND NUCLEIC ACID CONSTRUCTS CAPABLE OF SAME

(75) Inventor: Alon Chen, Rehovot (IL)

(73) Assignee: Yeda Research and Development Co. Ltd. at the Weizmann Institute of Science, Rehovot (IL)

(21) Appl. No.: 12/934,689

(22) PCT Filed: Mar. 25, 2009
(86) PCT No.: PCT/IL09/00341

§ 371 Date: Sep. 27, 2010

Related U.S. Application Data
(60) Provisional application No. 61/064,929, filed on Apr. 3, 2008.

(51) Int. Cl. A61K 31/7088; A61P 25/00; A61P 35/00; A61P 25/28; A61P 25/16; A61P 25/06; A61P 31/04; A61P 31/10; A61P 25/24; A61P 25/18; A61P 25/22; A61P 25/20; A61P 25/30; A61P 15/08; C12N 15/85

(52) U.S. Cl. 514/44 R; 435/320.1

(57) ABSTRACT

A nucleic acid construct and construct system are disclosed. The nucleic acid construct and system comprise a regulatory sequence which regulates inducible expression of a polypeptide of interest, the regulatory sequence comprising a choroid plexus specific promoter, with the proviso that the choroid plexus specific promoter is not a transthyretin promoter. Pharmaceutical compositions comprising same and uses thereof are also disclosed.

### FIELD AND BACKGROUND OF THE INVENTION

The present invention, in some embodiments thereof, relates to methods of expressing polypeptides in the brain and nucleic acid constructs for same.

Pharmacological administration of synthetic peptides or recombinant proteins into the brain ventricles is a common method employed for exploring physiological and behavioral functions of novel or known gene products. The need for direct administration into the ventricles rises from the presence of the Blood-Brain-Barrier (BBB), which prevents a non-selective transport of peptides and proteins from the periphery into the CNS.

Current solutions for administration of peptides or secreted proteins to the CNS, for short-term acute administration, include a stereotaxic injection of the tested protein into the ventricular space (intracerebroventricular injection, ICVI) or for chronic administration, an ICV microinjection pump. These methods rely heavily on the solubility and half-life of the injected substance; require chemical or in-vitro synthesis of the administered ligand, and may require different purification procedures. In addition, the ICVI is difficult to use in studies requiring repeated or prolonged administration of the ligand, since the microinjection pump is limited by the capacity of the reservoir and requires complex surgical procedures for installation and manipulation. Furthermore, the current procedures require an extensive handling of the experimental animals, which may present disturbances for several behavioral or physiological setups.

Two recently developed systems present a different strategy for crossing the BBB. The Receptor Mediated Transport (RMT) uses monoclonal peptide-mimetic antibodies (MAb's) to help large molecules to cross the BBB [Pardridge W M. Pharm Res 2007; 24(9): 1733-44]. These MAb's are either conjugated or fused to a peptide of interest and then use endogenous receptors to gain entry across the BBB. While this systems offers easy non-invasive administration (intravenous) it offers only a transient effect, it lacks specificity to the brain (i.e. it will act on all tissues that present the receptor) and may elicit an immune response. Spencer and Verma [Proc Natl Acad Sci USA. 2007; 104(18): 4594-9] suggest another technique based on the RMT strategy, which utilizes a lentiviral vector encoding the fused MAb-peptide. The virus is injected intra-peritoneal (i.p.) infecting mainly the liver and spleen. Infected cells secrete the protein into the blood stream where it crosses the BBB via transcytosis. While this model offers the promise of a chronic administration to the brain without the need for invasive or repeated administration its ability to reach physiological effective levels of the fused peptide in the brain remains to be tested.

The Choroid plexus (CP) is a secretory epithelial tissue suspended at multiple loci in the cerebroventricular system. In addition to manufacturing the CSF, it performs a diversity of homeostatic functions to stabilize the interstitial environment of neurons. Kidney, liver, and immune-type functions have been ascribed to the CP. The CP-CSF nexus furnishes micronutrients, growth factors, and neurotrophins to neuronal networks, making it a valuable target for the development of pharmacological agents that will distribute along similar CSF pathways to targets in the brain.

The choroidal epithelium has structural and functional properties that distinguish it from the cerebral endothelium of the BBB. Tight junctions which contain the protein occludin are “spot welds” at the apical zone of neighboring cells block the passage from blood to CSF. CP cells are highly vascularized with fenestrated capillaries that provide a perfusion rate 5-10 times that of the mean Cerebral Blood Flow (CBF). This fact coupled with a microvili structure of the apical lamina and abundant ion transporters and mitichondria enable the CP to play a role in CSF secretion and reabsorption.

The CSF effectively distributes native and foreign compounds, thus substances presented ICV have a larger volume of distribution in CNS than those injected intrathecally above the brain and spinal cord.

The CP strongly and specifically (in the brain) expresses the β splice variant of the type 2 CRF receptor [Chen A., Mol Endocrinol 2005; 19: 441-458]. Other genes which are specifically expressed in the CP include GPR125 [Pickering et al. BMC Neurosci. 2008; 9: 97] and transthyretin [Costa et al., Molecular and Cellular Biology, January, 1988, p. 81-90; Chen et al., The EMBO Journal, Vol. 9, No. 3, p 869-878, 1990].

U.S. Patent Application 20080280356 teaches nucleic acid constructs comprising transthyretin promoters.

### SUMMARY OF THE INVENTION

According to an aspect of some embodiments of the present invention there is provided a nucleic acid construct comprising a regulatory sequence which regulates inducible expression of a polypeptide of interest, said regulatory sequence comprising a choroid plexus specific promoter, with the proviso that said choroid plexus specific promoter is not a transthyretin promoter.

According to some embodiments of the invention, the nucleic acid construct comprises a polynucleotide sequence encoding said polypeptide of interest.

According to some embodiments of the invention, the regulatory sequence comprises a tetracycline response element.

According to some embodiments of the invention, the nucleic acid construct further comprises an additional polynucleotide sequence encoding a transactivator positioned under a control of said choroid plexus specific promoter, said transactivator in combination with an inducer are for regulating transcription of said polypeptide of interest.

According to an aspect of some embodiments of the present invention there is provided a nucleic acid construct system comprising a first nucleic acid construct comprising a first regulatory sequence and a second expression construct including a second polynucleotide sequence encoding a transactivator positioned under the transcriptional control of a choroid plexus specific promoter, wherein said transactivator activates said first regulatory sequence to direct transcription of a polypeptide of interest operatively linked to said first regulatory sequence, and wherein a transactivating activity of said transactivator is controlled by an inducer.

According to some embodiments of the invention, the nucleic acid construct system further comprises the polynucleotide sequence encoding the polypeptide of interest.

According to an aspect of some embodiments of the present invention there is provided a pharmaceutical composition comprising a polynucleotide comprising a nucleic acid sequence encoding a therapeutic polypeptide operatively linked to a choroid plexus specific promoter.

According to some embodiments of the invention, the choroid plexus specific promoter is a β splice variant of the type 2 corticotrophin releasing factor receptor (CRFR2β) promoter or a G protein-coupled receptor 125 (GPR125) promoter.

According to some embodiments of the invention, the choroid plexus specific promoter is selected from the group consisting of a β splice variant of the type 2 corticotrophin releasing factor receptor (CRFR2β) promoter, a G protein-coupled receptor 125 (GPR125) promoter and a transthyretin promoter.

According to some embodiments of the invention, the pharmaceutical composition comprises a nucleic acid construct system comprising a first nucleic acid construct comprising a first regulatory sequence and a second expression construct including a second polynucleotide sequence encoding a transactivator positioned under the transcriptional control of a choroid plexus specific promoter, wherein said transactivator activates said first regulatory sequence to direct transcription of a polypeptide of interest operatively linked to said first regulatory sequence, and wherein a transactivating activity of said transactivator is controlled by an inducer.

According to some embodiments of the invention, the pharmaceutical composition comprises a nucleic acid construct comprising a regulatory sequence which regulates inducible expression of a polypeptide of interest, said regulatory sequence comprising a choroid plexus specific promoter, with the proviso that said choroid plexus specific promoter is not a transthyretin promoter.

According to some embodiments of the invention, the nucleic acid construct further comprises an additional polynucleotide sequence encoding a transactivator positioned under a control of said choroid plexus specific promoter, said transactivator in combination with an inducer are for regulating transcription of said polypeptide of interest.

According to some embodiments of the invention, the nucleic acid construct system or pharmaceutical composition further comprises a third nucleic acid construct including a third polynucleotide sequence encoding a polypeptide processing enzyme positioned under the transcriptional control of said first regulatory sequence.

According to some embodiments of the invention, the inducer upregulates said transactivating activity of said transactivator.

According to some embodiments of the invention, the inducer downregulates said transactivating activity of said transactivator.

According to some embodiments of the invention, the inducer is a tetracycline.

According to some embodiments of the invention, the inducer is capable of crossing a blood brain barrier.

According to some embodiments of the invention, the inducer is doxycycline.

According to an aspect of some embodiments of the present invention there is provided a method of expressing a polypeptide of interest in a brain of a subject, the method comprising administering to the subject a polynucleotide comprising a nucleic acid sequence encoding the polypeptide, said polynucleotide being operatively linked to a choroid plexus specific promoter, thereby delivering the polypeptide to the brain of the subject.

According to some embodiments of the invention, the polypeptide of interest is a therapeutic agent.

According to an aspect of some embodiments of the present invention there is provided a kit for expressing a polynucleotide in a brain of a subject, the kit comprising the nucleic acid construct of the present invention or the nucleic acid construct system of the present invention and in a separate container said inducer.

According to an aspect of some embodiments of the present invention there is provided a method of treating a brain disorder or disease in a subject in need thereof, the method comprising administering to the subject a therapeutically effective amount of the pharmaceutical composition of the present invention, thereby treating the brain disorder or disease in the subject.

According to some embodiments of the invention, the therapeutic agent is a polypeptide selected from the group consisting of an enzyme, a growth factor, and an antibody.

According to some embodiments of the invention, the therapeutic agent is a neuropeptide.

According to some embodiments of the invention, the neuropeptide is selected from the group consisting of Oxytocin, Vasopressin, Corticotropin releasing hormone (CRH), Growth hormone releasing hormone (GHRH), Luteinizing hormone releasing hormone (LHRH), Somatostatin growth hormone release inhibiting hormone, Thyrotropin releasing hormone (TRH), Neurokinin a (substance K), Neurokinin β, Neuropeptide K, Substance P, β-endorphin, Dynorphin, Met- and leu-enkephalin, Neuropeptide tyrosine (NPY), Pancreatic polypeptide, Peptide tyrosine-tyrosine (PYY), Glucogen-like peptide-1 (GLP-1), Peptide histidine isoleucine (PHI), Pituitary adenylate cyclase activating peptide (PACAP), Vasoactive intestinal polypeptide (VIP), Brain natriuretic peptide, Calcitonin gene-related peptide (CGRP) (α- and β-form), Cholecystokinin (CCK), Galanin, Islet amyloid polypeptide (IAPP), Melanin concentrating hormone (MCH), ACTH, α-MSH, Neuropeptide FF, Neurotensin, Parathyroid hormone related protein, Agouti gene-related protein (AGRP), Cocaine and amphetamine regulated transcript (CART)/peptide, Endomorphin-1 and -2, 5-HT-moduline, Hypocretins/orexins Nociceptin/orphanin FQ, Nocistatin, Prolactin releasing peptide, Secretoneurin and Urocortin.

According to an aspect of some embodiments of the present invention there is provided a cell comprising the nucleic acid construct system of the present invention.

According to an aspect of some embodiments of the present invention there is provided a cell comprising the nucleic acid construct of the present invention.

According to some embodiments of the invention, the polynucleotide is comprised in a nucleic acid construct system comprising a first nucleic acid construct comprising a first regulatory sequence and a second expression construct including a second polynucleotide sequence encoding a transactivator positioned under the transcriptional control of a choroid plexus specific promoter, wherein said transactivator activates said first regulatory sequence to direct transcription of a polypeptide of interest operatively linked to said first regulatory sequence, and wherein a transactivating activity of said transactivator is controlled by an inducer.

According to some embodiments of the invention, the polynucleotide is comprised in a nucleic acid construct comprising a regulatory sequence which regulates inducible expression of a polypeptide of interest, said regulatory sequence comprising a choroid plexus specific promoter, with the proviso that said choroid plexus specific promoter is not a transthyretin promoter.

According to some embodiments of the invention, the method further comprises administering an inducer to the subject.

According to some embodiments of the invention, the method further comprises administering a polynucleotide sequence encoding a polypeptide processing enzyme positioned under a transcriptional control of a regulatory sequence.

According to some embodiments of the invention, the polynucleotide is administered to a brain ventricle of the subject.

According to some embodiments of the invention, the polynucleotide is administered to a spinal cord of the subject.

According to some embodiments of the invention, the inducer is administered orally.

According to some embodiments of the invention, the inducer is doxycycline.

According to some embodiments of the invention, the brain disease or disorder is selected from the group consisting of brain tumor, neuropathy, Alzheimer's disease, Parkinson's disease, Huntington's disease, amyotrophic lateral sclerosis, motor neuron disease, traumatic nerve injury, multiple sclerosis, acute disseminated encephalomyelitis, acute necrotizing hemorrhagic leukoencephalitis, dysmyelination disease, mitochondrial disease, migrainous disorder, bacterial infection, fungal infection, stroke, aging, dementia, schizophrenia, depression, manic depression, anxiety, panic disorder, social phobia, sleep disorder, attention deficit, conduct disorder, hyperactivity, personality disorder, drug abuse, infertility and head injury.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B are schematic representations of the lentiviral based system for inducible over-expression of peptides/proteins of interest in the cerebro-spinal-fluid (CSF). In FIG. 1B, infected choroid plexus (CP) cells are colored in green. Addition of Doxycycline induces the cells to synthesize and secrete the protein of interest into the cerebro-spinal-fluid.

FIGS. 1C-E are schematic representation of the constructs used in the system according to an embodiment of the present invention.

FIGS. 2A-I are photographs illustrating choroid plexus specific expression of CRFR2β. FIG. 2A. RT-PCR showing CRFR2β expressed by CP but not by hypothalamus (HT). FIGS. 2B-C. Dark-field photomicrographs showing in-situ hybridization for CRFR2β at the choroid plexus. FIGS. 2D-I. In-vivo demonstration of GFP expression by infected CP cells. Brain slices of mice ICV injected with “effector” virus were DAPI stained (FIGS. 2D, G), and GFP visualized (FIGS. 2E, H). A merge of DAPI and GFP images show GFP expression at CP cells, but not at cells surrounding the ventricle (FIGS. 2F, I). CP—choroid plexus, LV—lateral ventricle.

FIGS. 3A-F are photographs illustrating the RT-PCR results for various processing enzymes in the choroid plexus. Furin—(FIG. 3A); Prohormone convertase 1 (PC1)—(FIG. 3B); Prohormone convertase 2 (PC2)—(FIG. 3C); carboxypeptidase E (CPE)—(FIG. 3D); peptidyl-glycine-alpha-amidating-mono-oxygenase 1 (PAM1)—(FIG. 3E), and S16 control (FIG. 3F).

FIG. 3G is a schematic diagram of an inducible lentiviral construct for expression of additional processing enzymes.

FIGS. 3H-K are photomicrographs of HEK-293T cells infected with the three different viruses showing expression of RFP (FIG. 3H), GFP (FIG. 3I), and BFP (FIG. 3J), merged in FIG. 3K, proving the feasibility of a triple co-infection.

FIGS. 4A-N are photomicrographs illustrating inducible over-expression of mCRF in the cerebro-spinal-fluid (CSF) using a choroid plexus (CP) specific lentiviral based system. Brain sections were stained for the presence of GFP (Cy2) and mCRF (Cy3) with (FIGS. 4A-G) or without (FIGS. 4H-N) administration of the inducer doxycycline.

FIGS. 5A-D are graphs illustrating an increase in anxiety like behavior in C57B/6 mice conditionally over-expressing mCRF in the choroid plexus measured by the dark/light transfer test with or without the inducer doxycycline (Dox). Values are expressed as mean±SEM ** p<0.005, * p<0.05.

FIGS. 6A-D are graphs illustrating an increase in anxiety like behavior in C57B/6 mice conditionally over-expressing mCRF in the choroid plexus measured by open field test with or without the inducer doxycycline (Dox). Values are expressed as mean±SEM ** p<0.005, * p<0.05.

FIGS. 7A-B are graphs illustrating an increase in anxiety like behavior in C57B/6 mice conditionally over-expressing mCRF in the choroid plexus as measured by the elevated plus maze test with or without the inducer doxycycline (Dox). Values are expressed as mean±SEM ** p<0.005, * p<0.05.

FIG. 7C is a graph illustrating no change in anxiety in C57B/6 mice conditionally over-expressing mCRF in the choroid plexus as measured by the home cage locomotion with or without the inducer doxycycline (Dox). Values are expressed as mean±SEM ** p<0.005, * p<0.05.

FIGS. 8A-N are photomicrographs illustrating inducible over-expression of GnRH in the cerebro-spinal-fluid (CSF) using a choroid plexus (CP) specific lentiviral based system. Brain sections were stained for the presence of GFP (Cy2) and mGnRH (Cy3) with (FIGS. 8A-G) or without (FIGS. 8H-N) administration of the inducer doxycycline.

FIG. 9A is a graph illustrating the percent of animals showing an intact estrous cycle at pre-induced, induced and post-induced conditions. Values are expressed as mean±SEM ** p<0.0001.

FIG. 9B is a graph illustrating the estrous cycle stage of a representative mouse as determined by vaginal smears throughout the experiment. Values are expressed as mean±SEM ** p<0.0001.

### DESCRIPTION OF SPECIFIC EMBODIMENTS OF THE INVENTION

The present invention, in some embodiments thereof, relates to methods of expressing polypeptides in the brain and nucleic acid constructs for same.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The penetration of peripherally administered peptides into the central nervous system (CNS) is controlled by the blood-brain-barrier (BBB) rendering the intravenous administration of peptides to the CNS ineffective and creating a need for a bypass. Current methods for crossing the blood brain barrier rely mainly on mechanical approaches which involve direct infusion of peptides into the cerebro-spinal-fluid (CSF). Such methods are complex, costly and require constant invasive procedures.

The Choroid plexus (CP) is a secretory epithelial tissue constantly secreting cerebrospinal fluid. The current inventor(s) have conceived of using choroid plexus specific promoters in constructs to mediate transcription of secreted peptides for CNS delivery via the cerebrospinal fluid. Using such an approach, secreted polypeptides may reach extensive areas of the brain since the cerebrospinal fluid has a large volume of distribution. In addition, this approach allows for the chronic expression of polypeptides, whilst alleviating the need for expensive and complex micro-injection machinery and micropumps.

Whilst reducing the present invention to practice, the present inventors have generated lentiviral constructs comprising the type 2 CRF receptor (CRFR2β) promoter to mediate specific (and inducible) transcription of two secreted peptides in the choroid plexus for CNS delivery via the CSF: The corticotrophin releasing factor (CRF) and the gonadotrophin releasing hormone (GnRH), which are accountable for mediating the stress-induced behavioral changes and the intact estrous cycle, respectively.

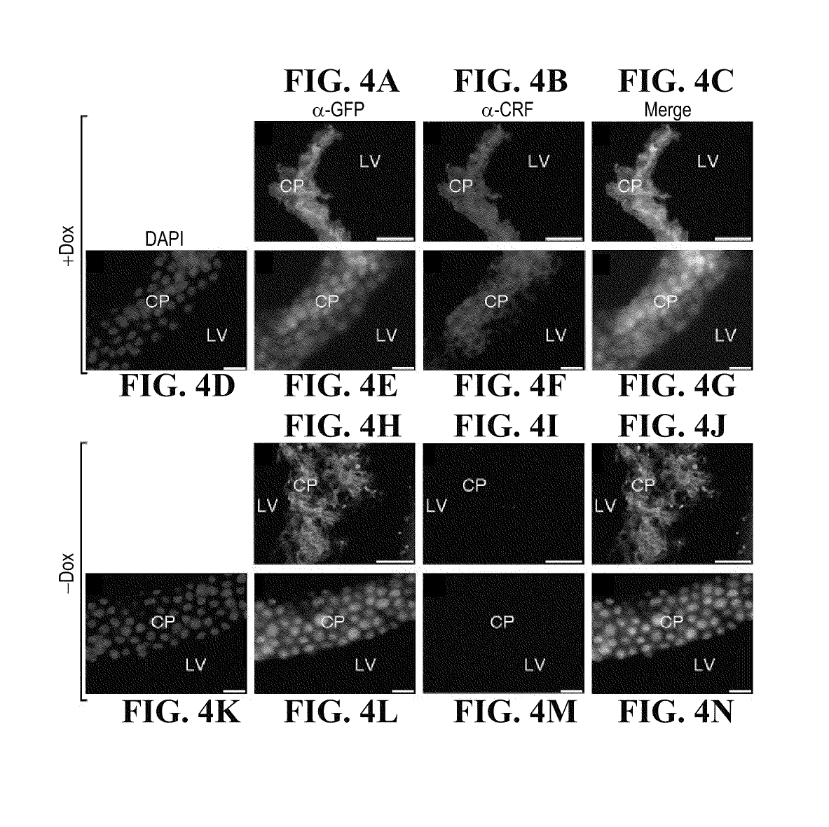

Using immunohistochemistry, the present inventors showed a specific and inducible over-expression of both CRF and GnRH in choroid plexus (CP) cells (FIGS. 4A-N and 8A-N, respectively).

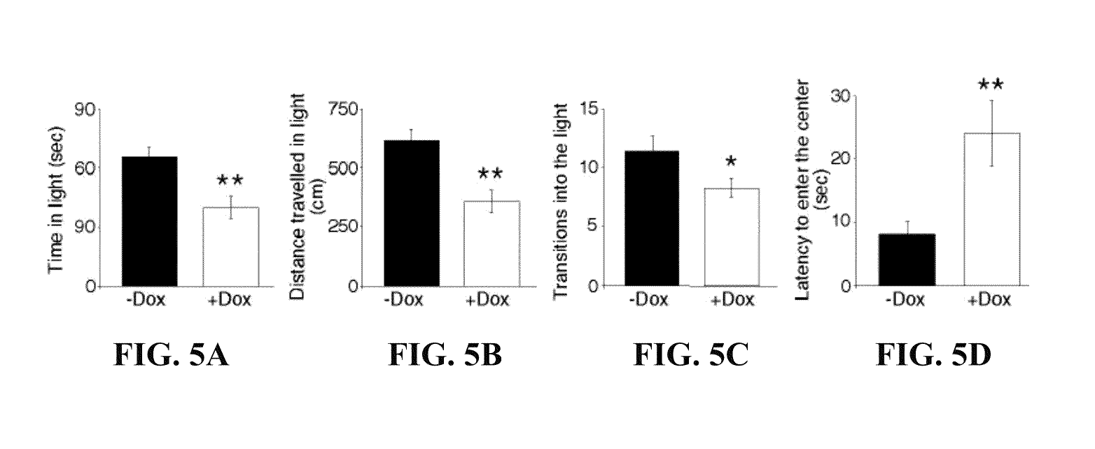

The present inventors injected the CRF over-expressing lentiviruses into mice and examined the effect using a battery of stress-related behavioral tests, in the presence or absence of the inducer, doxycycline. As illustrated in FIGS. 5A-D, 6A-D and 7A-C, mice under induced conditions demonstrated a significant increase in anxiety and depression-like behaviors, which corresponds to the established effects of central CRF over-expression.

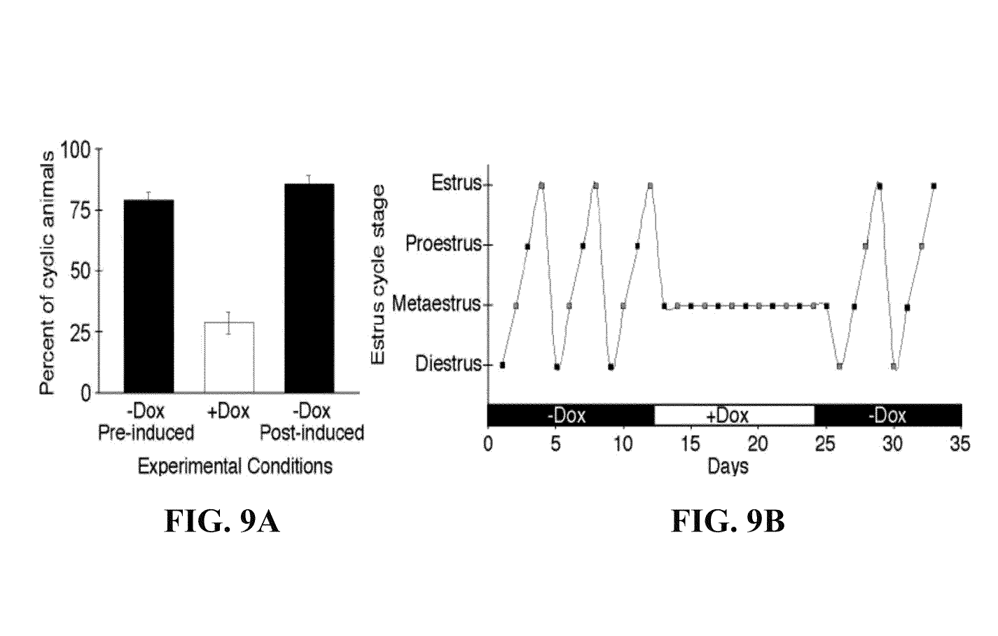

In addition, the present inventors injected GnRH over-expressing lentiviruses into female mice injected and detected an inducible and reversible disruption of their estrous cycle FIGS. 9A-B.

Thus, according to one aspect of the present invention there is provided a method of expressing a polypeptide of interest in a brain of a subject. The method comprises administering to the subject a polynucleotide comprising a nucleic acid sequence encoding the polypeptide, the polynucleotide being operatively linked to a choroid plexus specific promoter.

As used herein, the term “polypeptide” refers to a polymer of any length made up of natural amino acids only. Accordingly, the term polypeptide includes short peptides and full length proteins.

The polypeptide may be useful for research, diagnostic or therapeutic purposes, as further described below.

As used herein the term “subject” refers to a mammal (e.g. human, mouse, rat, rabbit, bovine, porcine, ovine, canine and feline).

As used herein, the phrase “choroid plexus specific promoter” refers to a polynucleotide sequence capable of directing expression of a polynucleotide sequence to which it is operably linked, to the cells of the choroid plexus and not to other cells (such as neuronal cells or ependymal cells).

Examples of choroid plexus specific promoters include, but are not limited to a β splice variant of the type 2 corticotrophin releasing factor receptor (CRFR2β) promoter (e.g. as set forth in SEQ ID NO: 19), a G protein-coupled receptor 125 (GPR125) promoter (as set forth in SEQ ID NO: 20) and a transthyretin promoter (e.g. as set forth in SEQ ID NO: 21).

According to one embodiment of this aspect of the present invention, the choroid plexus specific promoter is not a transthyretin promoter.

The Choroid plexus (CP) is a secretory epithelial tissue responsible for manufacturing the cerebrospinal fluid (CSF) suspended at multiple loci in the cerebroventricular system. The choroidal epithelium has structural and functional properties that distinguish it from the cerebral endothelium of the blood brain barrier. CP cells are highly vascularized with fenestrated capillaries that provide a perfusion rate 5-10 times that of the mean Cerebral Blood Flow (CBF). This fact coupled with a microvili structure of the apical lamina and the presence of abundant ion transporters and mitichondria enables the CP to play a role in CSF secretion and reabsorption.

According to one embodiment, the choroid plexus specific promoter sequence is placed 3′ to the polynucleotide sequence encoding the polypeptide of interest on a nucleic acid construct such that expression thereof is constitutive, but tissue specific.

According to another embodiment, the choroid plexus specific promoter sequence is situated relative to the polynucleotide sequence encoding the polypeptide of interest on a nucleic acid construct such that expression thereof is tissue specific, but also may be controlled in an exogenously regulatable fashion.

In order to ensure that the polypeptide of interest is expressed both specifically in cells of the choroid plexus and in an exogenously controllable fashion, a nucleic acid construct may be designed such that it comprises a polynucleotide encoding a transactivator under control of the choroid plexus specific promoter. The polynucleotide encoding the polypeptide of interest may be inserted in the same nucleic acid construct or in an additional construct under control of an inducible promoter. The transactivator in combination with an inducer act to regulate expression from the inducible promoter.

Inducible promoters suitable for use with the present invention are preferably response elements capable for directing transcription of the polynucleotide sequence so as to confer regulatable synthesis of the polypeptide of interest. A suitable response element can be, for example, a tetracycline response element (such as described by Gossen and Bujard (Proc. Natl. Acad. Sci. USA 89:5547-551, 1992); an ectysone-inducible response element (No D et al., Proc Natl Acad Sci USA. 93:3346-3351, 1996) a metal-ion response element such as described by Mayo et al. (Cell. 29:99-108, 1982); Brinster et al. (Nature 296:39-42, 1982) and Searle et al. (Mol. Cell. Biol. 5:1480-1489, 1985); a heat shock response element such as described by Nouer et al. (in: Heat Shock Response, ed. Nouer, L., CRC, Boca Raton, Ha., pp 167-220, 1991); or a hormone response element such as described by Lee et al. (Nature 294:228-232, 1981); Hynes et al. (Proc. Natl. Acad. Sci. USA 78:2038-2042, 1981); Klock et al. (Nature 329:734-736, 1987); and Israel and Kaufman (Nucl. Acids Res. 17:2589-2604, 1989). Preferably the response element is an ectysone-inducible response element, more preferably the response element is a tetracycline response element.

An exemplary system that may be used according to this embodiment of the present invention is a Tet-on expression system. In this system, the reverse tetracycline transactivator (rtTA) binds to the Tet Response Element (TRE) and activates transcription only in the presence of tetracycline or derivatives thereof.

When two constructs are used, the chimeric reverse transactivator (rtTA) (under control of the CP-specific promoter on a first construct), activates (in the presence of the inducer) transcription of the polypeptide of interest from a silent, inducible promoter (Tet response element; TRE) present on the second construct. When one construct is used the chimeric transactivator under control of the CP-specific promoter activates transcription of the polypeptide of interest from the silent inducible promoter all on the same construct (see for example Chtarto et al., Gene Therapy (2003) 10, 84-94).

Another exemplary construct that may be used according to this embodiment of the present invention is a Tet-off expression system. In this system, the tetracycline transactivator (tTA) under control of the CP-specific promoter, activates (in the absence of the inducer), transcription of the polypeptide of interest. In the presence of the inducer, the tTA loses its ability to bind the TRE, and expression of the polypeptide of interests is shut off.

The regimen for administration of the inducer is selected according to the construct (for example, tet-on or tet-off) and the type of expression required for the polypeptide of interest (continuous, intermittent etc.). According to one embodiment, the inducer is administered directly into the brain or through the spine. If the inducer is capable of crossing the blood brain barrier (e.g. doxycycline) it may be administered centrally (e.g. orally). Typically, the inducer is administered following administration of the constructs of the present invention.

The nucleic acid constructs of the present invention may also include one or more enhancers. Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for the present invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

Polyadenylation sequences can also be added to the nucleic acid construct in order to increase the translation efficiency of the polypeptide of interest expressed from the expression construct of the present invention. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for the present invention include those derived from SV40.

In addition to the elements already described, the nucleic acid construct of the present invention may typically contain other specialized elements intended to increase the level of expression of cloned polynucleotides or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The nucleic acid constructs may or may not include a eukaryotic replicon. If a eukaryotic replicon is present, then the vector is amplifiable in eukaryotic cells using the appropriate selectable marker. If the construct does not comprise a eukaryotic replicon, no episomal amplification is possible. Instead, the recombinant DNA integrates into the genome of the engineered cell, where the promoter directs expression of the desired polynucleotide.

The nucleic acid constructs of the present invention can further include additional polynucleotide sequences that allow, for example, the translation of several proteins from a single mRNA such as an internal ribosome entry site (IRES) and sequences for genomic integration of the promoter-chimeric polypeptide. For example a single expression construct can be designed and co-express two distinct polypeptides one the polypeptide of interest, and one a processing enzyme as further described herein below.

Examples of mammalian expression constructs include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression constructs containing regulatory elements from eukaryotic viruses such as retroviruses can also be used by the present invention. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

Viruses are specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell.

Recombinant viral vectors are useful for in vivo expression of transgenic polynucleotides since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

According to one embodiment, the constructs of the present invention are incorporated into lentiviruses. These viruses are advantageous because of their ability to integrate their DNA into the genome of mammalian non-dividing cells in-vivo.

It will be appreciated that the nucleic acid construct can be designed as a gene knock-in construct in which case it will lead to genomic integration of construct sequences, or it can be designed as an episomal expression vector.

In any case, the nucleic acid construct can be generated using standard ligation and restriction techniques, which are well known in the art (see Maniatis et al., in: Molecular Cloning: A Laboratory Manual, Cold Spring Harbor Laboratory, New York, 1982). Isolated plasmids, DNA sequences, or synthesized oligonucleotides are cleaved, tailored, and religated in the form desired.

Various methods can be used to introduce the nucleic acid constructs of the present invention into mammalian cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In order to circumvent the blood brain barrier, the constructs of the present invention may be administered directly into the brain (via the ventricle) or via the spinal cord (e.g. by an epidural catheter).

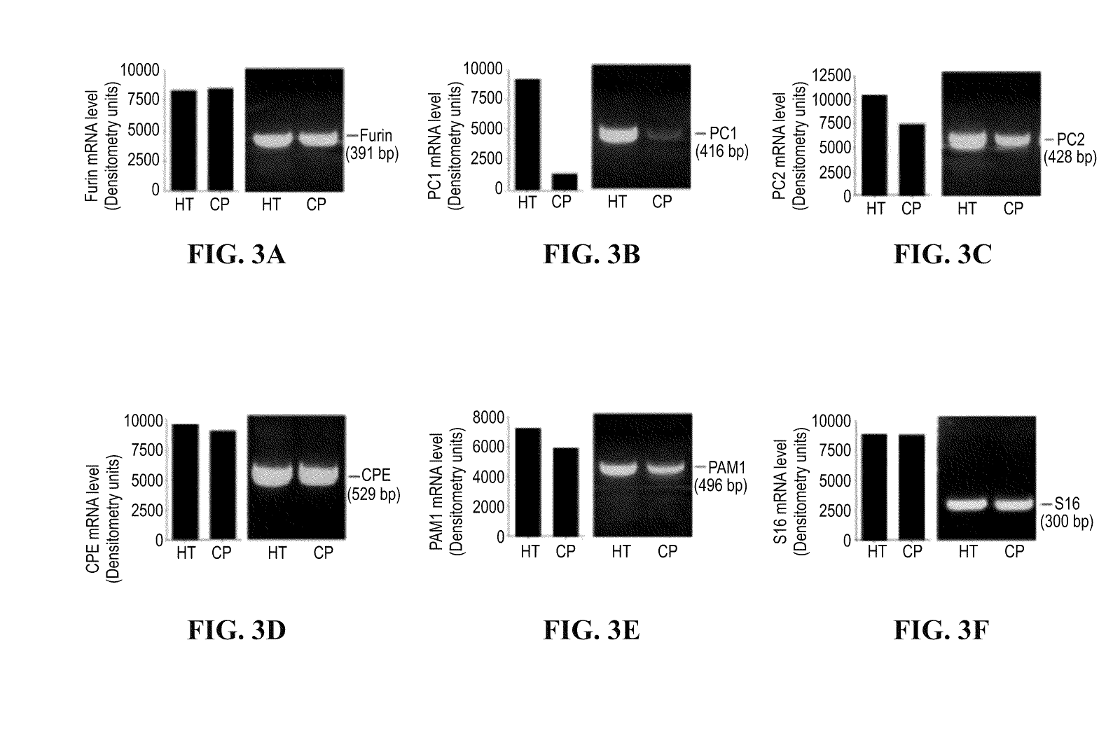

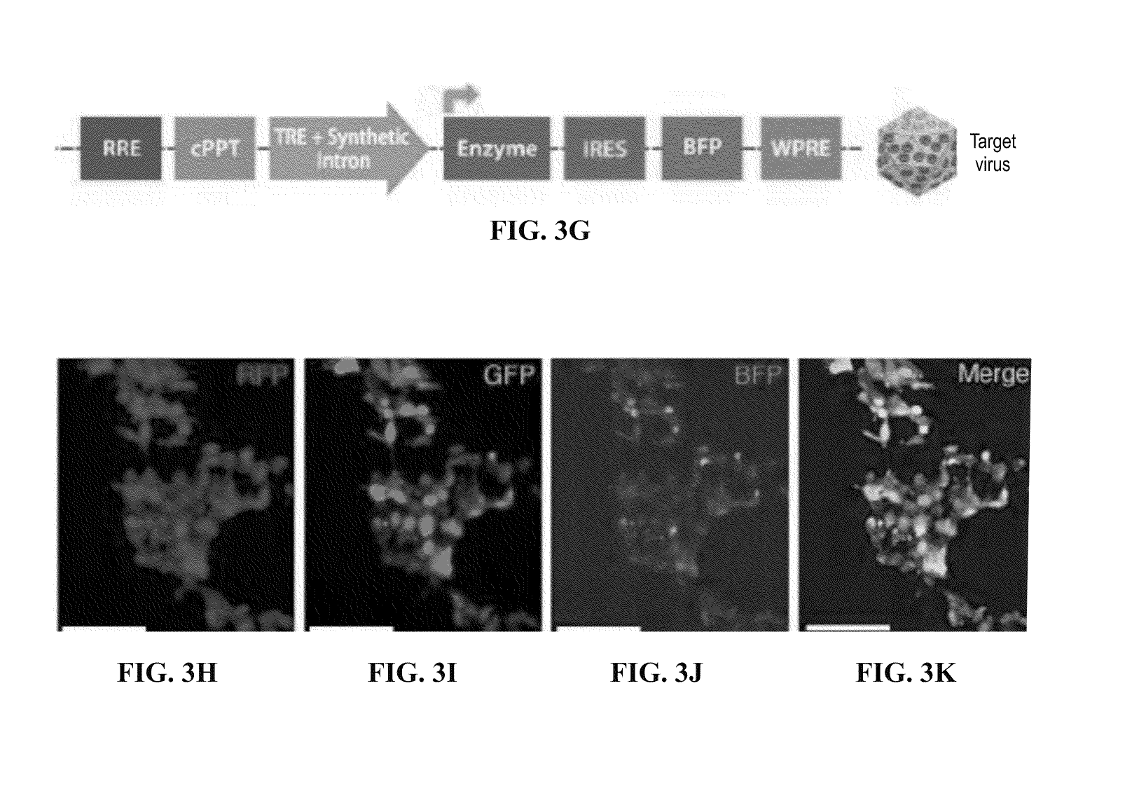

As many peptides/polypeptides require post-translational processing of a non-active precursor to become viable, it is necessary that the proper processing enzymes be expressed in the CP. Although the present inventors have shown that CP cells express a battery of processing enzymes necessary for precursor cleavage and amidation (FIGS. 3A-F), the present invention also contemplates expression of additional processing enzymes.

As used herein, the phrase “polypeptide processing enzyme” refers to an enzyme capable of cleaving and/or modifying a polypeptide.

According to one embodiment, the polypeptide processing enzyme is a neuropeptide processing enzyme.

Exemplary processing enzymes contemplated for expression by the present invention include, but are not limited to furin (EC 3.4.21.75), Prohormone convertase 1 (PC1) (EC 3.4.21.93) Prohormone convertase 2 (PC2) (EC 3.4.21.94), carboxypeptidase E (CPE) (EC 3.4.17.10) and peptidyl-glycine-alpha-amidating-mono-oxygenase 1 (PAM1) (EC 1.14.17.3)

Thus, according to an embodiment of the present invention the method of the present invention is effected by administering a polynucleotide sequence encoding a polypeptide processing enzyme positioned under a transcriptional control of a regulatory sequence in conjunction with the construct(s) encoding the polypeptide of interest. The polypeptide processing enzyme sequence may be present on the constructs described herein above or may be administered on a separate construct. The regulatory sequence for the polypeptide processing enzyme may be a constitutive promoter or an inducible promoter such as the ones described herein above.

According to an exemplary embodiment, the regulatory sequence is the identical regulatory sequence that is linked to the polypeptide of interest (e.g. the TRE). In such a system, the tetracycline inducer controls expression of both the processing enzyme and the polypeptide of interest. In this embodiment, the polypeptide processing enzyme construct may be administered to the subject prior to, concomitant with or following administration of the construct which encodes the polypeptide of interest, but prior to administration of the inducer.

As mentioned, the constructs of the present invention are used to express polypeptides of interest in the brain. Exemplary polypeptides contemplated by the present invention include neuropeptides, enzymes, structural polypeptides, growth factors and antibodies.

According to one embodiment, the constructs may be used to express secreted polypeptides in the cells of the CP. This would enable the polypeptides to be secreted into the cerebrospinal fluid and reach large areas of the brain.

The term “neuropeptides” as used herein, includes peptide hormones, peptide growth factors and other peptides. Examples of neuropeptides which can be used in accordance with the present invention include, but are not limited to Oxytocin, Vasopressin, Corticotropin releasing hormone (CRH), Growth hormone releasing hormone (GHRH), Luteinizing hormone releasing hormone (LHRH), Somatostatin growth hormone release inhibiting hormone, Thyrotropin releasing hormone (TRH), Neurokinin a (substance K), Neurokinin 13, Neuropeptide K, Substance P, β-endorphin, Dynorphin, Met- and leu-enkephalin, Neuropeptide tyrosine (NPY), Pancreatic polypeptide, Peptide tyrosine-tyrosine (PYY), Glucogen-like peptide-1 (GLP-1), Peptide histidine isoleucine (PHI), Pituitary adenylate cyclase activating peptide (PACAP), Vasoactive intestinal polypeptide (VIP), Brain natriuretic peptide, Calcitonin gene-related peptide (CGRP) (α- and β-form), Cholecystokinin (CCK), Galanin, Islet amyloid polypeptide (IAPP), Melanin concentrating hormone (MCH), Melanocortins (ACTH, α-MSH and others), Neuropeptide FF, Neurotensin, Parathyroid hormone related protein, Agouti gene-related protein (AGRP), Cocaine and amphetamine regulated transcript (CART)/peptide, Endomorphin-1 and -2, 5-HT-moduline, Hypocretins/orexins Nociceptin/orphanin FQ, Nocistatin, Prolactin releasing peptide, Secretoneurin and Urocortin.

According to another embodiment, the polypeptide is one that is expressed in the choroid plexus (CP)—for example a structural protein or an enzyme.

According to another embodiment, the polypeptide is one which comprises a detectable moiety. This may be useful for diagnostic imaging of choroid plexus disease. Polypeptides comprising detectable moieties are well known to those of skill in the art. They include, but are not limited to, bacterial chloramphenicol acetyl transferase (CAT), beta-galactosidase, green fluorescent protein (GFP) and other fluorescent protein, various bacterial luciferase genes, e.g., the luciferase genes encoded by Vibrio harveyi, Vibrio fischeri, and Xenorhabdus luminescens, the firefly luciferase gene FFlux, and the like.

It will be appreciated that the constructs of the present invention may also be used to express polynucleotide agents in the CP including RNA silencing agents such as siRNAs, microRNAs.

It will be appreciated that when the polypeptide of interest is a therapeutic polypeptide it may be useful for treating a disease.

Thus, according to another aspect of the present invention there is provided a method of treating a brain disorder or disease in a subject in need thereof. The method comprises administering to the subject a therapeutically effective amount of a therapeutic polypeptide using the constructs of the present invention.

Examples of brain disorders which may be treated or diagnosed by the agents of the present invention include, but are not limited to brain tumor, neuropathy, Alzheimer's disease, Parkinson's disease, Huntington's disease, amyotropic lateral sclerosis, motor neuron disease, traumatic nerve injury, multiple sclerosis, acute disseminated encephalomyelitis, acute necrotizing hemorrhagic leukoencephalitis, dysmyelination disease, mitochondrial disease, migrainous disorder, bacterial infection, fungal infection, stroke, aging, dementia, schizophrenia, depression, manic depression, anxiety, panic disorder, social phobia, sleep disorder, attention deficit, conduct disorder, hyperactivity, personality disorder, drug abuse, infertility and head injury.

According to one embodiment, the disease is one that is particular to the choroid plexus. Such diseases include (1) neoplasms (papilloma, leukaemia, meningioma, lymphoma and metastases) of the choroid plexus; (2) infections (bacterial, fungal and viral) of the choroid plexus; (3) cysts in the choroid plexus; (4) haemorrhage in the choroid plexus; (5) congenital abnormalities (Sturge-Weber syndrome, Klippel-Trenaunay-Weber syndrome and vascular malformations); and (6) non-infectious inflammatory disorders (xanthogranulomas, inflammatory pseudotumour, neurosarcoidosis, rheumatoid nodule and villous hypertrophy).

The constructs of the present invention can be provided to the individual per se, or as part of a pharmaceutical composition where it is mixed with a pharmaceutically acceptable carrier.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the constructs which are accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases. One of the ingredients included in the pharmaceutically acceptable carrier can be for example polyethylene glycol (PEG), a biocompatible polymer with a wide range of solubility in both organic and aqueous media (Mutter et al. (1979).

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

As mentioned, suitable routes of administration include direct injection into the brain or spinal cord (intrathecal).

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological salt buffer.

Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Pharmaceutical compositions suitable for use in context of the present invention include compositions wherein the active ingredients are contained in an amount effective to achieve the intended purpose. More specifically, a therapeutically effective amount means an amount of active ingredients effective to prevent, alleviate or ameliorate symptoms of disease or prolong the survival of the subject being treated.

Determination of a therapeutically effective amount is well within the capability of those skilled in the art.

For any preparation used in the methods of the invention, the therapeutically effective amount or dose can be estimated initially from in vitro assays. For example, a dose can be formulated in animal models and such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. [See e.g., Fingl, et al., (1975) “The Pharmacological Basis of Therapeutics”, Ch. 1 p. 1].

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

It will be appreciated that the constructs of the present invention can be provided to the individual with additional active agents to achieve an improved therapeutic effect as compared to treatment with each agent by itself. In such therapy, measures (e.g., dosing and selection of the complementary agent) are taken to adverse side effects which may be associated with combination therapies.

Compositions including the preparation of the present invention formulated in a compatible pharmaceutical carrier may also be prepared, placed in an appropriate container, and labeled for treatment of an indicated condition.

Compositions of the present invention may, if desired, be presented in a pack or dispenser device, such as an FDA approved kit, which may contain one or more unit dosage forms containing the active ingredient. The kit may also contain an inducer, such as the ones described herein above. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

It is expected that during the life of a patent maturing from this application other relevant choroid specific promoters may be discovered and the scope of the term choroid specific promoter is intended to include all such promoters a priori. In addition, it is expected that other inducible construct systems will be developed and the present invention contemplates the use of all.

As used herein the term “about” refers to ±10%

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, an and “the” include plural references unless the context clearly dictates otherwise. For example, the term “a compound” or “at least one compound” may include a plurality of compounds, including mixtures thereof.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion. Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

Design and Construction of Tet-on Lentiviral Vectors for Inducible Choroid Plexus-Specific Expression

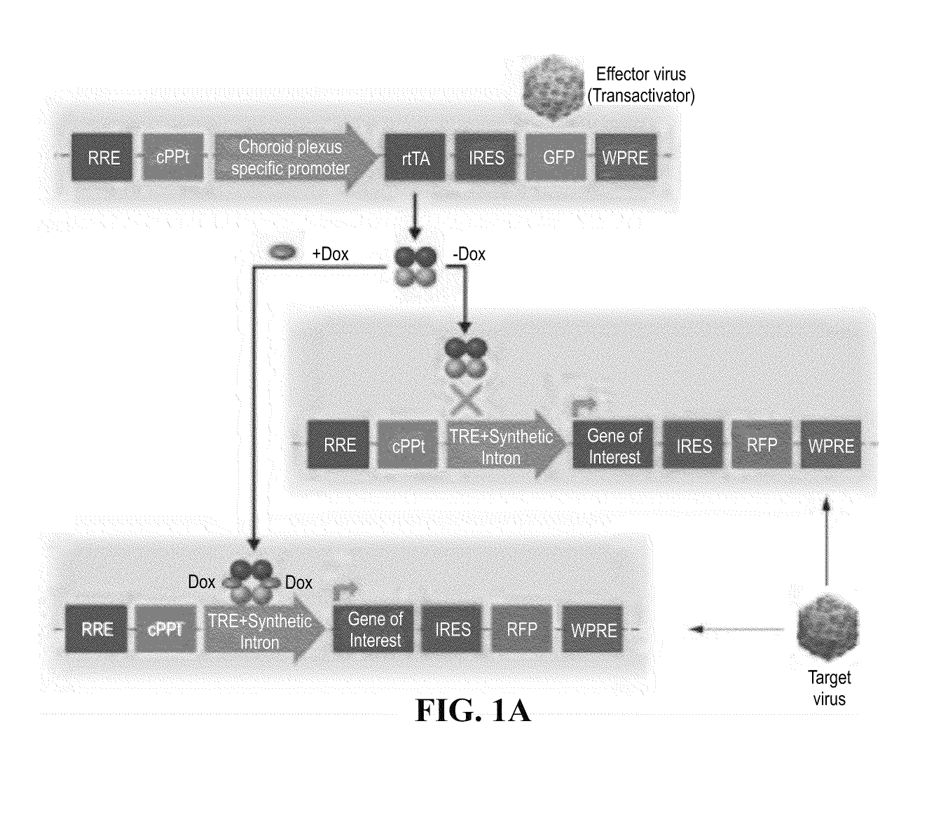

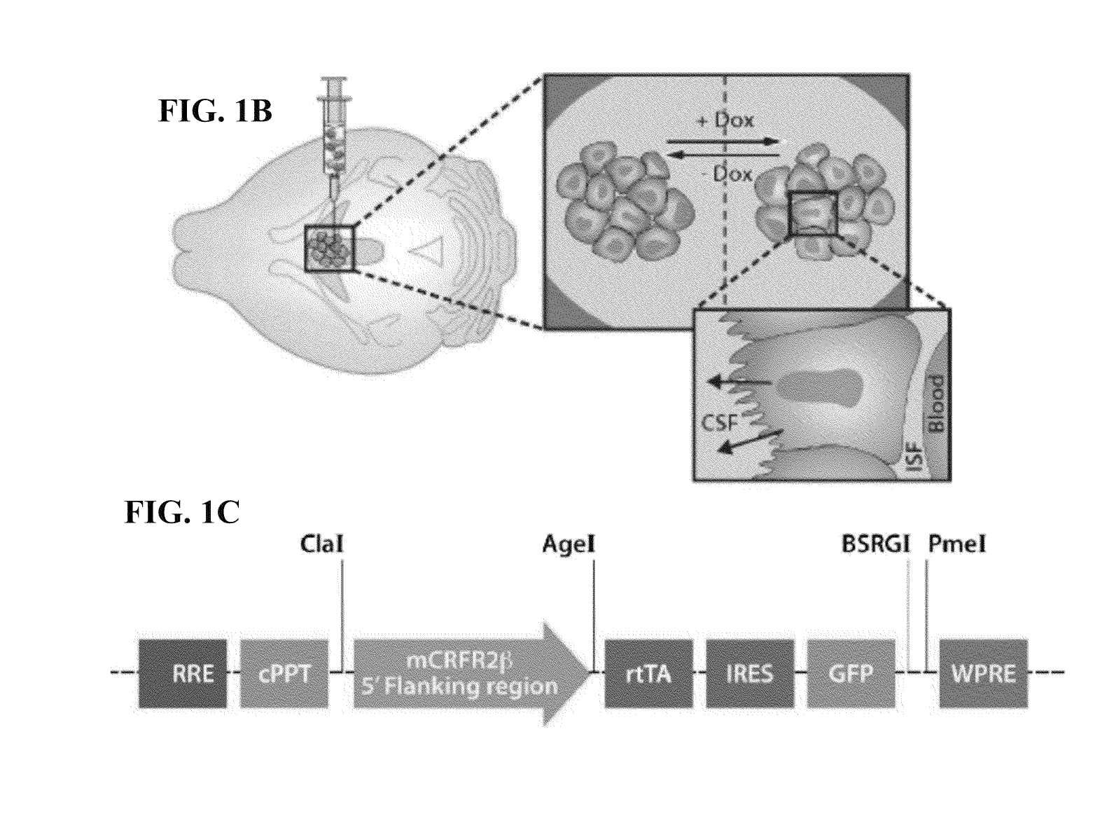

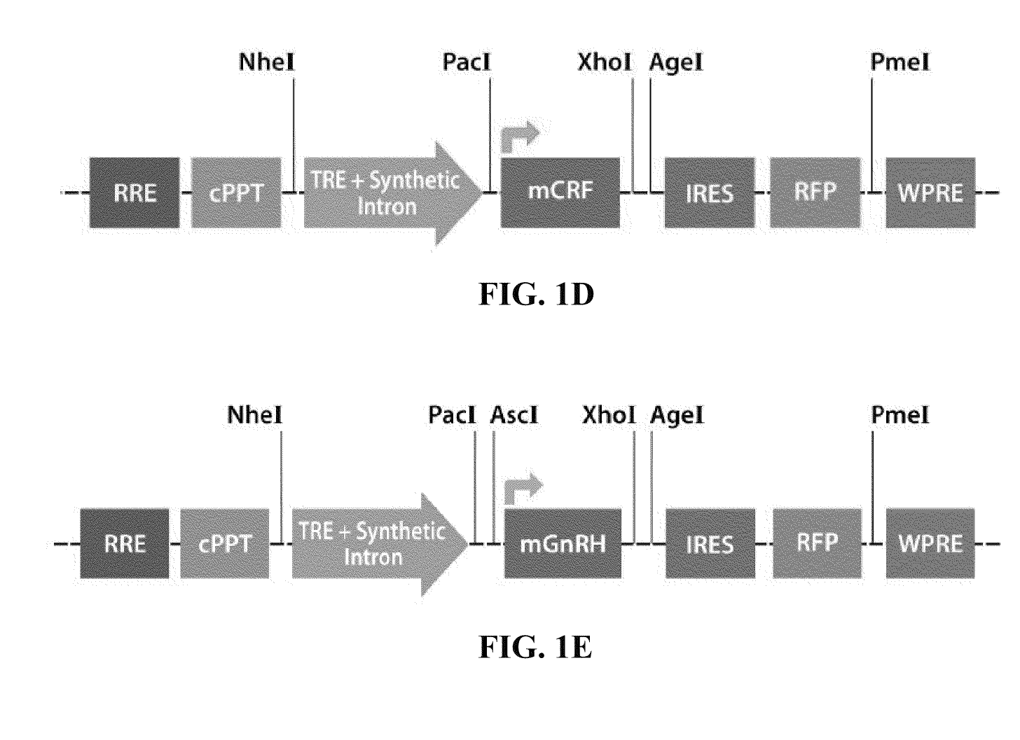

In order to generate an inducible system for over-expression of peptides or secreted proteins in the CSF, choroid plexus-specific Tet-On lentiviral vectors were designed. The Tet-On gene expression system enables regulation of gene expression in a precise, reversible and quantitative manner. The Tet system uses the chimeric transactivator (tTA) to activate transcription of the gene of interest from a silent promoter (FIG. 1A). Gene expression is controlled through the interaction of two components, the product of an ‘effector’ transgene carried by one virus, acting on a ‘target’ transgene carried by a second virus (FIG. 1A). In the Tet-On system, the reverse tTA (rtTA) binds to the Tet Response Element (TRE) and activates transcription only in the presence of tetracycline or derivatives (FIG. 1A). Doxycycline (Dox) was selected as the inducer as it has been demonstrated to cross the BBB. Administration of Dox containing drinking water results in secretion of the final processed gene product into the CSF (FIG. 1B).

To generate inducible over-expression of peptide by the choroid plexus cells, choroid plexus cells were infected with a mixture of both ‘effector’ and ‘target’ viruses. To achieve tissue specific transgene expression, the 5′-flanking region of the CRFR2β gene, which was previously demonstrated to be expressed specifically in choroid plexus cells [Chen et al., Mol Endocrinol 2005; 19: 441-458], was selected to drive the transcription of the reverse transactivator (rtTA).

Materials and Methods

All constructs were assembled by standard cloning methods and confirmed by DNA sequencing. Choroid Plexus specific Tet-On lentiviral vectors were designed based on the pCSC-SP-PW-IRES-GFP lentiviral vector plasmid.

“Effector plasmid”: the reverse tetracycline transactivator (rtTA) coding sequence was PCR-amplified from a rtTA-expressing construct and ligated into the AgeI and PstI sites of pCSC-SP-PW-IRES-GFP resulting in the pCSC-SP-PW-rtTA-IRES-GFP lentiviral construct. Next, a ˜3 kb sequence, up-stream to the CRFR2 gene first exon (CRFR2βpro) (SEQ ID NO: 19) was PCR-amplified from mouse purified DNA. The amplified sequence was then inserted into the pCSC-SP-PW-rtTA-IRES-GFP construct using ClaI and AgeI sites, replacing the CMV promoter and resulting in the pCSC-SP-PW-CRFR2βpro-rtTA-IRES-GFP lentiviral construct, as illustrated in FIG. 1C.

“Target plasmid”: a coding sequence for monomeric RFP was subcloned into a pIRES plasmid (Clonetech) using XbaI and SalI sites. The IRES-RFP sequence was PCR amplified and transferred into the pCSC-SP-PW-IRES-GFP using AgeI and Bsp1407I sites, replacing the RES-GFP sequence. Next, a multiple cloning site (MCS) from the pGL3-basic plasmid (Promega) was PCR amplified with primers carrying 5′ and 3′ AgeI sites, and a ClaI site down-stream to the 5′ AgeI. The MCS was ligated into the pCSC-SP-PW-IRES-RFP plasmid, using the AgeI site, downstream to the CMV promoter. The resulting plasmid was digested with ClaI to excise the CMV promoter sequence.

A Tetracycline responsive element (TRE) was PCR amplified from a TRE-containing plasmid inserted using a HindIII site upstream to a mouse CRF (mCRF) cDNA sequence (GenBank accession no. NM_{—}205769 (SEQ ID NO: 22)) in a pCDNA3-mCRF plasmid. Following that, the TRE-mCRF sequence was subcloned into the pCSC-SP-PW-IRES-RFP plasmid using the NheI and XhoI sites in the added MCS resulting in the pCSC-SP-PW-TRE-mCRF-IRES-RFP lentiviral construct—see FIG. 1D. Mouse GnRH (mGnRH) cDNA was PCR amplified from mouse hypothalamic cDNA using the following specific oligonucleotide primers: 5′-GCTGGCCTCTTTGCTAA-3′ (SEQ ID NO: 17) and 5′-AGTGCATCTACATCTTCTTCTG-3′ (SEQ ID NO: 18) corresponding to nucleotides 20-36 and 335-356 respectively (GenBank accession no. BC116897 (SEQ ID NO: 23)), and TA-cloned into a pCR-TOPO2.1 plasmid (Invitrogen). The mGnRH sequence (GenBank accession no. BC116897) was then inserted into the pCSC-SP-PW-TRE-mCRF-IRES-RFP plasmid, using PacI and XhoI sites replacing the CRF sequence and resulting in the pCSC-SP-PW-TRE-mGnRH-IRES-RFP plasmid—see FIG. 1E.

Production of lentiviral vectors: Recombinant lentiviruses were produced by transient transfection in HEK293T cells, as described previously [Naldini L, et al., Science 1996; 272: 263-267; Naldini L, et al., Proc Natl Acad Sci U S A., 1996; 93: 11382-11388; Pfeifer A, et al., Proc Natl Acad Sci USA. 2000; 97: 12227-12232; Chen A, J. Neurosci. 2006; 26: 5500-5510]. Briefly, infectious lentiviruses were harvested at 48 and 72 hours post-transfection, filtered through 0.45 μm-pore cellulose acetate filters and concentrated by ultracentrifugation. Vector concentrations were analyzed using eGFP or RFP fluorescence in HEK293T cells infected with serial dilutions of the recombinant lentivirus.

Animals: 7 weeks old wild-type C57BL/6J male mice and ICR female mice (Harlan, Jerusalem, Israel) were used for the experimental procedures. Mice were housed in a temperature-controlled room (22° C.±1) on a reverse 12 hours light/dark cycle. Food and water were given ad libitum. All experimental protocols were approved by the Institutional Animal Care and Use Committee of The Weizmann Institute of Science.

Surgical procedure: Animals were anesthetized with 10 μg ketamine, 0.8 μg xylazine, 4 μg acepromazine per gr. body weight, intraperitoneally, and placed in a Angle Two stereotaxic instrument (myNeuroLab, St. Louis, Mo., USA). 2 μl of concentrated lentiviral vector preparation was injected into the left lateral ventricle of mice, using a 26s-gauge blunt-tip needle Hamilton microsyringe (Hamilton, Reno, Nev., USA), at a rate of 0.5 μl/min. Injection coordinates relative to bregma were as follows: AP −0.22 mm; ML −1.15 mm; DV −2.06 mm.

RT-PCR analysis: primers are listed in Table 1, herein below

In situ-hybridization: Brain slices were analyzed 14 days following infection at 4° C.

Results

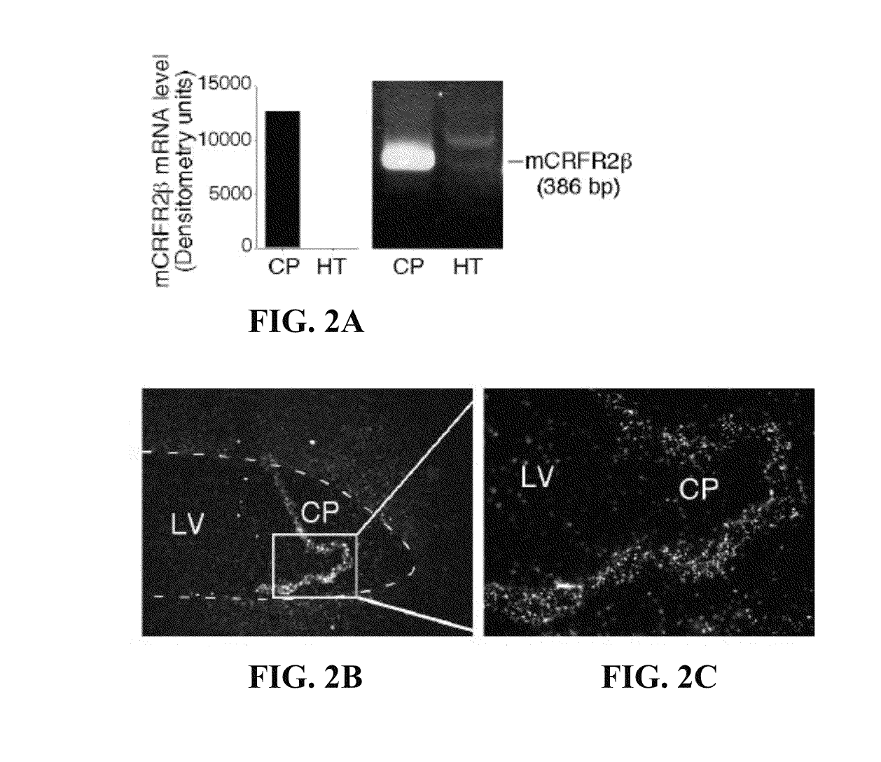

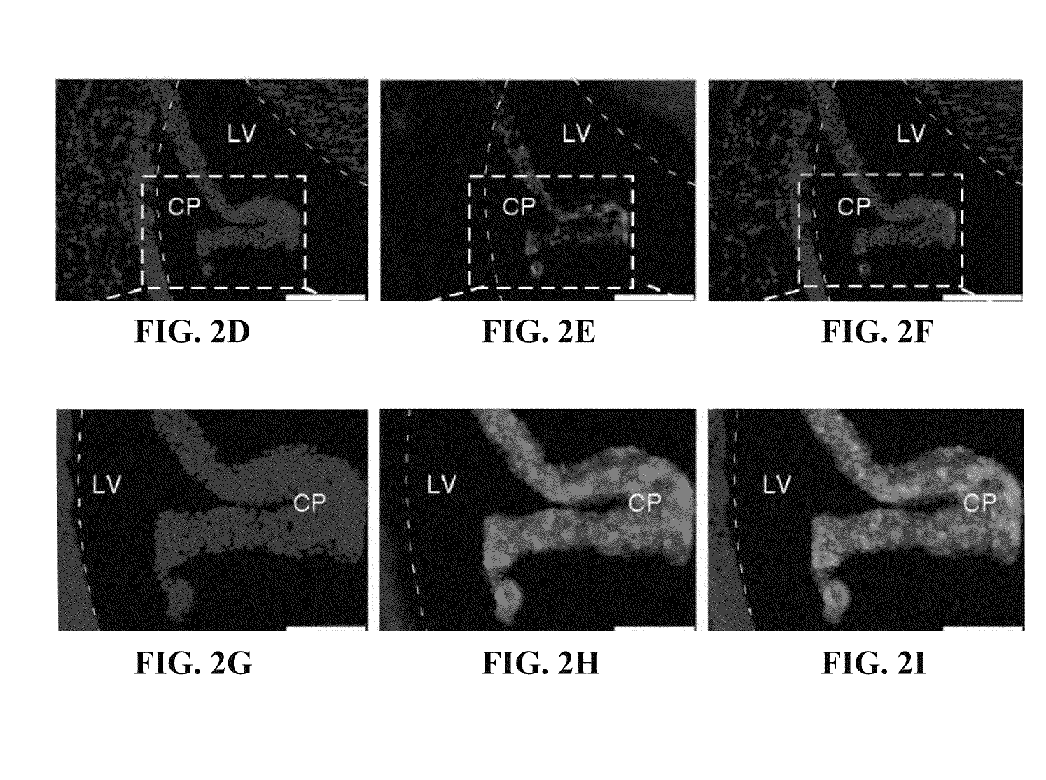

To confirm the specificity of central CRFR2β expression to the CP, total RNA was extracted from mouse CP and hypothalamic tissues and was reverse-transcribed to generate cDNAs. The cDNA products were used as templates for semiquantitative RT-PCR analysis, using specific primers for mouse CRFR2β (FIG. 2A). The RT-PCR data demonstrate expression of CRFR2β that is abundant and restricted to the CP tissue (FIG. 2A). To further demonstrate the mRNA distribution of mouse CRFR2β in the CP cells, a specific in situ probe was used and a positive hybridization signal for mouse CRFR2β was found only in the CP (FIGS. 2B-C).

Intracerebroventricular injection of the CP-specific lentiviruses show GFP expression specifically by the CP cells (FIGS. 2D-I). Co-staining with the nuclear marker, DAPI, clearly demonstrates that only the CP-cells and not the ependymal cells (cells which line the brain ventricles) express GFP (FIGS. 2D-F). Higher magnification images demonstrate the ability of these lentiviruses to infect the CP-cells with high efficiency (FIGS. 2G-I).

### Example 2

### Verification of Neuropeptide Processing Enzymes Expression by Choroid Plexus Cells

Many secreted neuropeptides require post-translational processing of their precursors in order to become an active peptide. In order to verify whether choroid plexus cells are capable of such post-translational modifications PCR amplification was used for the detection of neuropeptide processing enzymes expression in choroid plexus cells.

Materials and Methods

Choroid plexus was collected from twenty C57B/6 mice, using dissecting microscope, and RNA was extracted using TRI-reagent (Sigma) according to the manufacturer's recommendations. Total RNA was reverse transcribed (SuperScript II, Invitrogen) to generate a choroid plexus cDNA pool. The cDNA product was used as template for semiquantitative PCR analysis using specific primers as summarized in Table 1. The expression of ribosomal protein S16 served as internal control.

TABLE 1

| | Primer sequence | Primer sequence | | Amplicon |
|---|---|---|---|---|
| Enzyme | (sense) | (anti-sense) | Annealing | size |
| Furin | 5′GACCGCGGCCT | 5′GGCCGCCCCT | 58° C. | 391 bp |
| | CATCTCCTACA 3′ | CTTCACTCTG 3′ | | |
| | SEQ ID NO: 1 | SEQ ID NO: 2 | | |
| Proprotein | 5′TCAGGGAATG | 5′CCAAATCCAA | 53.5° C. | 416 bp |
| Convertase type 1 | GGGGTCGTCA 3′ | ATCGGCTGTTCA 3′ | | |
| (PC1) | SEQ ID NO: 3 | SEQ ID NO: 4 | | |
| Proprotein | 5′CAACGCGACCA | 5′AGAGGCAGAG | 59° C. | 428 bp |
| Convertase type 2 | GGAGAGGAGACC 3′ | ACGGGGAGGA | | |
| (PC2) | SEQ ID NO: 5 | AGG 3′ | | |
| | | SEQ ID NO: 6 | | |
| Peptidylglycine | 5′AAAGAAGCCG | 5′CACGCGTTAA | 52° C. | 496 bp |
| Alpha-amidating | AGGCAGTTGTTGA 3′ | AGGGAAAGGA | | |
| Mono-oxygenase | SEQ ID NO: 7 | ATCT 3′ | | |
| type 1 (PAM) | | SEQ ID NO: 8 | | |
| Carboxypeptidase | 5′CGCCATCAGC | 5′CAGATTGGCA | 60° C. | 529 bp |
| E (CPE) | AGAATCTACA 3′ | GAAAGCACAA 3′ | | |
| | SEQ ID NO: 9 | SEQ ID NO: 10 | | |
| Transthyretin | 5′GCTTCCCTTC | 5′TCTCTCAATTC | 56° C. | 444 bp |
| (TTR) | GACTCTTCCT 3′ | TGGGGGTTG 3′ | | |
| | SEQ ID NO: 11 | SEQ ID NO: 12 | | |
| Corticotropin | 5′CTGGAACCTC | 5′GGGGCCCTGG | 56° C. | 386 bp |
| releasing factor | ATCACCACCT 3′ | TAGATGTAGT 3′ | | |
| receptor type 2 | SEQ ID NO: 13 | SEQ ID NO: 14 | | |
| beta (CRFR2β) | | | | |
| 16S ribosomal | 5′TGCGGTGTGGA | 5′GCTACCAGGC | 56° C. | 300 bp |
| RNA | GCTCGTGCTTGT 3′ | CTTTGAGATGGA 3′ | | |
| | SEQ ID NO: 15 | SEQ ID NO: 16 | | |

Results

Most neuropeptides are derived from larger biologically inactive polypeptide precursors and require post-translational processing to become biologically active. Specific proteases/endopeptidases are thought to process precursors during transit through the ER/golgi secretory pathway. Following cleavage, the residual basic amino acids are removed by an exopeptidase (carboxypeptidase B/H/E), which is often followed by other post-translational modifications such as glycosylation, sulphation and amidation to obtain full bioactivity.

To ensure that the target proteins will be properly processed by the CP cells, total RNA isolated from the mouse choroid plexus was screened for a set of key processing enzymes known to expressed by neuroendocrine neurons and to be necessary for the proper processing of most neuropeptides.

Semi-quantitative RT-PCR analysis of the paired basic amino acid cleaving enzyme Furin (FIG. 3A) and the exopeptidase E (CPE), which responsible for the removal of an amino acid from the end of a polypeptide chain (FIG. 3D), showed similar expression levels in the CP cells, compared with the positive control tissue obtained from the mouse hypothalamus.

The mRNA levels of the prohormone convertase 2 (PC2) (FIG. 3C) and the peptidylglycine alpha-amidating monooxygenase 1 (PAM1), which catalyze peptides to active alpha-amidated products (FIG. 3E), were only slightly reduced compared with the levels in the hypothalamic tissue. The PC1 expression levels, although detected, were significantly lower in the CP tissue than in hypothalamic tissue (FIG. 3B).

To allow the experimental system to be applied for peptides requiring processing enzymes and cofactors not expressed by the CP tissue, a third lentiviral DNA construct was designed to express a blue florescent protein (BFP) reporter, in an inducible manner (FIG. 3G). Infection of HEK-293T cells with the three reporter viruses, showed the feasibility of a triple-infection as can be seen in the fluorescent reporters visualization (FIG. 3H).

### Example 3

### Over-Expression of Corticotrophin-Releasing Factor and Anxiogenic Behavior

Materials and Methods

Animals and Housing

Animals: 7 week old wild-type C57BL/6J male mice and ICR female mice (Harlan, Jerusalem, Israel) were used for the experimental procedures. Mice were housed in a temperature-controlled room (22° C.±1) on a reverse 12 hours light/dark cycle. Food and water were given ad libitum. All experimental protocols were approved by the Institutional Animal Care and Use Committee of The Weizmann Institute of Science.

Surgical procedure: Animals were anesthetized with 10 μg ketamine, 0.8 μg xylazine, 4 μg acepromazine per gr. body weight, intraperitoneally, and placed in a Angle Two stereotaxic instrument (myNeuroLab, St. Louis, Mo., USA). 2 μl of concentrated lentiviral vector preperation was injected into the left lateral ventricle of mice, using a 26s-gauge blunt-tip needle Hamilton microsyringe (Hamilton, Reno, Nev., USA), at a rate of 0.5 μl/min. Injection coordinates relative to bregma were as follows: AP −0.22 mm; ML −1.15 mm; DV −2.06 mm.

Following a one week recovery period, mice were subjected to behavioral and physiological studies as described below. At the end of the tests, mice were anesthetized and perfused with phosphate buffered 4% paraformaldehyde.

Male mice (C57B/6, Harlan, Israel. n=16) were injected into the lateral ventricle with 2 μl of pCSC-SP-PW-CRFR2βpro-rtTA-IRES-GFP and pCSC-SP-PW-TRE-mCRF-IRES-RFP lentivirus cocktail.

Following a week recovery period mice were subjected to the following tests: open field, dark/light transfer box and elevated plus maze (EPM) followed by a home cage locomotion analysis. Animals were then given a 0.5 mg/ml doxycycline, 0.2% sucrose solution as drinking water for 4 days before repeating the same set of tests. To avoid experimental artifacts due to repetition, the EPM test was performed on animals only once (half group under each condition). Prior to sacrifice, half the group was kept on tap water and half on dox-containing water.

Central administration of CRF has been demonstrated to increase anxiogenic-like behavior. To evaluate the behavioral effect of central over-expression of CRF in the present model, mice were tested for anxiety related behaviors, under both induced and non-induced conditions using the following tests:

Light/dark transfer test: The light/dark transfer test apparatus and experimental conditions were as previously described [Chen et al., J. Neurosci. 2006; 26: 5500-5510]. The light/dark transfer test takes advantage of the natural conflict of a rodent between the exploration of a novel environment and the aversive properties of a large, brightly lit open field. A greater amount of time spent in the light compartment and a greater number of transitions are indicative of decreased anxiety-like behavior. The activity and transitions were quantified with a video tracking system (VideoMot2; TSE Systems, Bad Hamburg, Germany).

Open field test: The open field test apparatus and experimental conditions was as previously described [Chen et al., J. Neurosci. 2006; 26: 5500-5510]. Time (sec) spent in the center, number of visits to center, ambulation (total distance traveled), latency to enter center (sec) and rearings were quantified with a video tracking system (VideoMot2; TSE Systems, Bad Hamburg, Germany).

Elevated plus maze (EPM) test: The EPM apparatus and experimental conditions are as previously described [Chen et al., J. Neurosci. 2006; 26: 5500-5510; Lister R. G. Psychopharmacology 1987; 92: 180-185]. Anxiolytic and anxiogenic drugs respectively increase or decrease relative exploration of the open arms [Lister, supra]. Therefore, the number of entries into and the time spent on the open arms were expressed as a percentage of the total number of arm entries and test duration, respectively.

Locomotor activity: To control for the possibility of behavioral effects originating from differences in ambulatory movement, locomotor activity of mice was examined over a 48 hr period, which proceeded a few days of habituation. Mice were single housed in specialized home cages and locomotion was measured using the InfraMot system (TSE Systems, Bad Hamburg, Germany).

Immunohistochemistry for in-vivo validation of lentiviruses: The procedure used for immunohistochemistry is described in [Chen et al., J. Neurosci. 2006; 26: 5500-5510]. Briefly, mice were anesthetized with chloral hydrate (350 mg/kg, ip) and perfused with 4% paraformaldehyde fixative. Coronal 30 μm thick sections throughout the brain were prepared for immunofluorecence localization of GFP (rtTA virus) or CRF immunoreactivity.

Sections were washed (5 minutes×3) with PBS and incubated over night at 4° C. with PBS containing 0.2% triton, monoclonal α-GFP antibody (Chemicon) (1:1000) and rabbit α-CRF (1:1000) serum. Following PBS washes (5 minutes x3) sections were incubated for 2 hours at room temperature with Cy2 α-mouse (Jackson ImmunoResearch) (1:2000) and Cy3 α-rabbit (Jackson ImmunoResearch) (1:2000) secondary antibodies. Sections were washed with PBS (5 minutes×3), mounted on slides and visualized using an Olympus IX81 fluorescence microscope.

Statistical analysis: Results for behavioral tests of mice conditionally over-expressing CRF were calculated as mean values±SEM, and were analyzed using JMP 7 software (SAS, NC, USA). Student t-test was used to compare doxycycline induced and control groups for the elevated plus maze test. Paired t-test was used to compare between doxycycline induced and control conditions for the dark/light transfer test and the open field test.

### Results

To demonstrate the functionality of the established system the over-expression of corticotrophin-releasing factor (CRF), known to modulate anxiety-like behavior was studied.

A mixture of the effector and CRF-target lentiviruses were injected ICV to C57BL′6 mice. Brains were collected under induced (+Dox) or non-induced (−Dox) conditions, and processed for immunohistochemistry. Immunohistochemical analysis of brain slices showed a choroid plexus specific staining for GFP under both induced and non-induced conditions (FIGS. 4A, E, H and L) and a specific Dox-dependent staining for mouse CRF (FIGS. 4B, F, I and M).

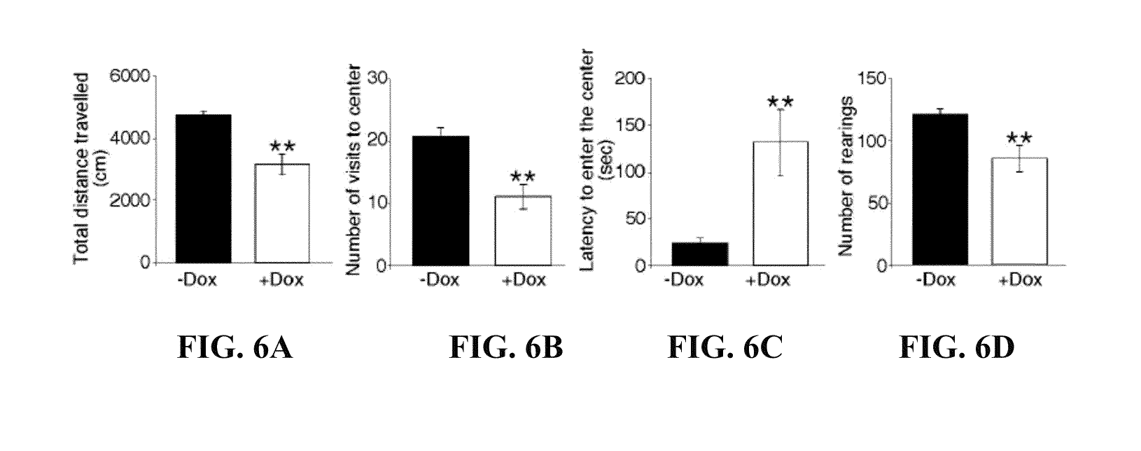

To evaluate the anxiety-like behavior of mice conditionally over-expressing CRF at the choroid plexus, a set of related behavioral tests were performed, with or without the presence of the inducer Dox. Three days following doxycycline induction, mice showed increase in anxiety-like behavior measured by the light/dark transfer test, with significantly less time spent in the light compartment, reduced exploration of light area, fewer transitions to the light compartment and increase in the latency to first enter the light compartment (FIGS. 5A-D). Results from the open field test were consistent with the results obtained from the light/dark transfer test. Mice over-expressing CRF showed a reduced number of entries to the center, a greater latency entering the center, decrease in rearing events and lower exploratory behavior as measured by the shorter distance traveled during the test (FIGS. 6A-D).

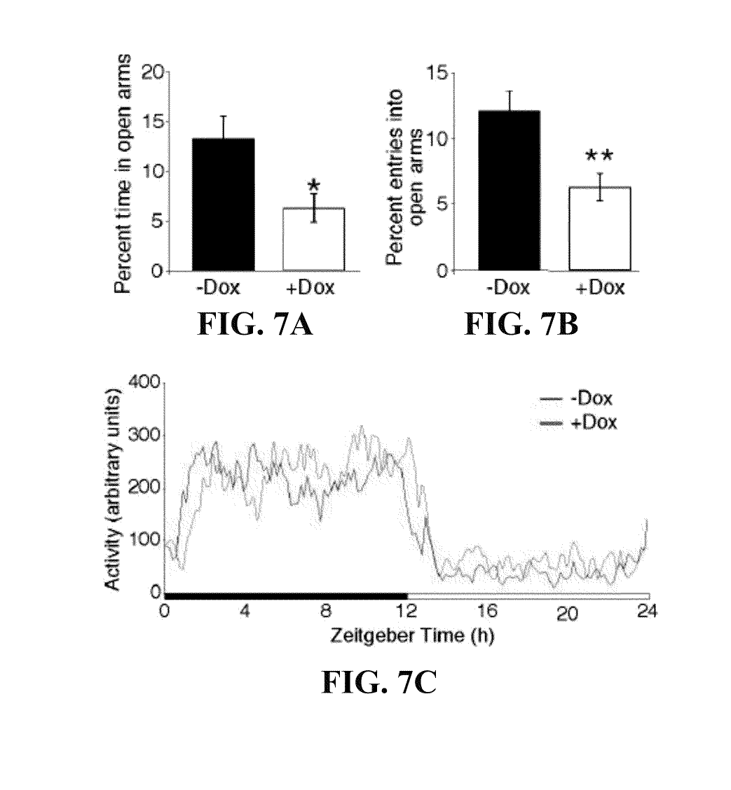

In accordance with the open field and dark/light transfer tests, the results from the elevated plus maze test show significant differences between the control and the induced group of mice. CRF over-expressing mice show a decrease in the percent of time spent in the open arm and the percent of entries into open arms (FIGS. 7A-B). No significant differences were found between the experimental groups in their home cage locomotor activity (FIG. 7C), suggesting that the observed phenotype is not a consequence of locomotion deficit but rather a genuine change in the mice anxiety-like behavior. These results clearly demonstrate that mice treated with Dox and over express CRF acutely, display increased levels of anxiety-like behavior as expected for mice over-expression CRF.

### Example 4

### Over-Expression of Gnrh and Estrus Cycle Modulation

Materials and Methods

Animals and Housing

Female mice (ICR, Harlan, Israel. n=30) were injected into the lateral ventricle with 2 μl of pCSC-SP-PW-CRFR2βpro-rtTA-IRES-GFP and pCSC-SP-PW-TRE-GnRH-IRES-RFP lentivirus cocktail. Following a two weeks recovery period vaginal smears were taken for estrous cycle determination every other day for 6 weeks. At day 16 animals were given a solution of 0.5 mg/ml doxycycline and 0.2% sucrose as drinking water. Following two more weeks of vaginal smears, animals were switched back to tap water and no smears were taken for one week to allow the doxycycline to clear. Then, vaginal smears were resumed for two additional weeks.

Reproductive physiology tests conducted in mice conditionally over-expressing GnRH.

GnRH is the central hypothalamic hormone regulating reproduction. The pulse-timing and concentration levels of GnRH are critical for the maintenance of gonadal steroidogenesis, normal reproductive function and estrous cycle. Chronic, high concentrations of GnRH induce regulatory changes that lead to gonadal hypoactivity and cessation or abnormalities of the estrous cycle.

Estrous cycle determination: The murine estrous cycle is 4-5 days long and its four stages are termed proestrus, estrus, metestrus, and diestrus. The stage of estrous was determined by cytological evaluation of vaginal smears as follows: a smear consisting almost exclusively of leukocytes depicted diestrus; a thin smear of equal numbers of leukocytes and elongated nucleated epithelium indicated proestrus; large cornified epithelial cells were exclusively found in estrus; and metestrus was marked by a thick smear composed of equal numbers of nucleated epithelial cells and leukocytes.

Vaginal smears from mature virgin female ICR mice (12-14 weeks old) were obtained once in two days at 10:30 am. Only those mice undergoing normal estrous cycle changes for at least three consecutive 4-day cycles were included in the study.

Mice were sacrificed at the stage of estrus/diestrus. To validate the accuracy of stage determination, vaginal smears were again obtained prior to sacrificing the animals.

Immunohistochemistry for in-vivo validation of lentiviruses: The procedure used for immunohistochemistry is described in [Chen et al., J. Neurosci. 2006; 26: 5500-5510]. Briefly, mice were anesthetized with chloral hydrate (350 mg/kg, ip) and perfused with 4% paraformaldehyde fixative. Coronal 30 μm thick sections throughout the brain were prepared for immunofluorecence localization of GFP (rtTA virus) or GnRH immunoreactivity.

Sections were washed (5 minutes×3) with PBS and incubated over night at 4° C. with PBS containing 0.2% triton, monoclonal α-GFP antibody (Chemicon) (1:1000) and rabbit α-GnRH (1:1000) serum. Following PBS washes (5 minutes×3) sections were incubated for 2 hours at room temperature with Cy2 α-mouse (Jackson ImmunoResearch) (1:2000) and Cy3 α-rabbit (Jackson ImmunoResearch) (1:2000) secondary antibodies. Sections were washed with PBS (5 minutes×3), mounted on slides and visualized using an Olympus IX81 fluorescence microscope.

Statistical analysis: Paired t-test was used to compare between doxycycline induced and non induced conditions in the estrous cycle determination experiment.

Results

To demonstrate the functionality of the established system the over-expression of gonadotropin-releasing factor (GnRH), known to modulate the estrus cycle was studied.

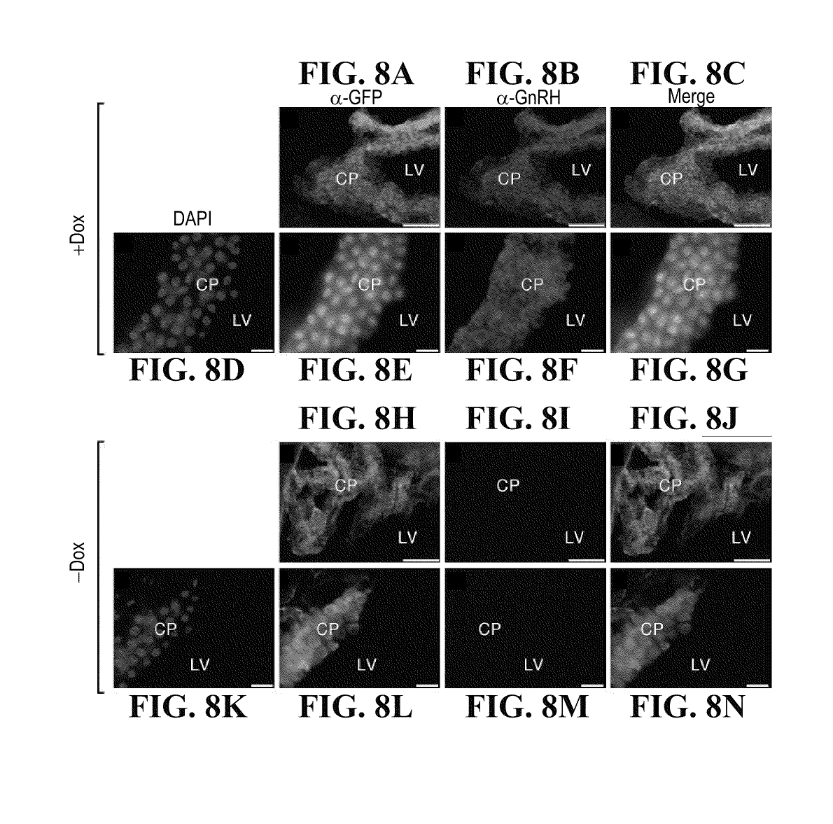

mGnRH was cloned into the “target” vector and both “effector” and “target” viruses were ICV injected into the lateral ventricle of female ICR mice. Following a recovery period, vaginal smears were taken for estrous cycle determination every other day. After 14 days, the animals were given dox in drinking water. Following two more weeks of estrous cycle determination, animals were switched back to regular drinking water monitored for an additional two weeks. Mice were sacrificed under induced or non-induced conditions, and their brains were processed for immunohistochemistry. This analysis showed a choroid plexus specific staining for GFP under both induced and non-induced conditions (FIGS. 8A, E, H and L) and a dox-dependent staining for mGnRH (FIGS. 8B, F, I and M). Estrous cycle determination showed that while most of the injected animals showed an adherence to a normal four-day estrous cycle under non-induced conditions, dox administration has reversibly disrupted the cycle, significantly reducing this adherence. Following removal of dox from drinking water a most animals re-established an intact 4 day cycle (FIG. 9A). FIG. 9B shows the estrous cycle of a representative injected mouse, throughout the experiment.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A nucleic acid construct comprising a regulatory sequence which regulates inducible expression of a polypeptide of interest, said regulatory sequence comprising a choroid plexus specific promoter, with the proviso that said choroid plexus specific promoter is not a transthyretin promoter.

2. The nucleic acid construct of claim 1, comprising a polynucleotide sequence encoding said polypeptide of interest.

3. The nucleic acid construct of claim 1, wherein said regulatory sequence comprises a tetracycline response element.

4. The nucleic acid construct of claim 1, further comprising an additional polynucleotide sequence encoding a transactivator positioned under a control of said choroid plexus specific promoter, said transactivator in combination with an inducer are for regulating transcription of said polypeptide of interest.

5. A nucleic acid construct system comprising a first nucleic acid construct comprising a first regulatory sequence and a second expression construct including a second polynucleotide sequence encoding a transactivator positioned under the transcriptional control of a choroid plexus specific promoter, wherein said transactivator activates said first regulatory sequence to direct transcription of a polypeptide of interest operatively linked to said first regulatory sequence, and wherein a transactivating activity of said transactivator is controlled by an inducer.

6. The nucleic acid construct system of claim 5, further comprising said polynucleotide sequence encoding said polypeptide of interest.

7. A pharmaceutical composition comprising a polynucleotide comprising a nucleic acid sequence encoding a therapeutic polypeptide operatively linked to a choroid plexus specific promoter.

8. The nucleic acid construct of claim 1, wherein said choroid plexus specific promoter is a β splice variant of the type 2 corticotrophin releasing factor receptor (CRFR2β) promoter or a G protein-coupled receptor 125 (GPR125) promoter.

9-18. (canceled)

19. A method of expressing a polypeptide of interest in a brain of a subject, the method comprising administering to the subject a polynucleotide comprising a nucleic acid sequence encoding the polypeptide, said polynucleotide being operatively linked to a choroid plexus specific promoter, thereby delivering the polypeptide to the brain of the subject.

20. The nucleic acid construct of claim 2, wherein said polypeptide of interest is a neuropeptide.

21. (canceled)

22. A method of treating a brain disorder or disease in a subject in need thereof, the method comprising administering to the subject a therapeutically effective amount of a pharmaceutical composition which comprises a polynucleotide comprising a nucleic acid sequence encoding a therapeutic polypeptide operatively linked to a choroid plexus specific promoter, thereby treating the brain disorder or disease in the subject.

23. (canceled)

24. The pharmaceutical composition of claim 7, wherein said therapeutic agent is a neuropeptide.

25-31. (canceled)

32. The method of claim 19, wherein said polynucleotide is administered to a brain ventricle of the subject.

33. The method of claim 19, wherein said polynucleotide is administered to a spinal cord of the subject.

34-35. (canceled)

36. The method of claim 22, wherein the brain disease or disorder is selected from the group consisting of brain tumor, neuropathy, Alzheimer's disease, Parkinson's disease, Huntington's disease, amyotrophic lateral sclerosis, motor neuron disease, traumatic nerve injury, multiple sclerosis, acute disseminated encephalomyelitis, acute necrotizing hemorrhagic leukoencephalitis, dysmyelination disease, mitochondrial disease, migrainous disorder, bacterial infection, fungal infection, stroke, aging, dementia, schizophrenia, depression, manic depression, anxiety, panic disorder, social phobia, sleep disorder, attention deficit, conduct disorder, hyperactivity, personality disorder, drug abuse, infertility and head injury.

37. The method of claim 19, wherein said polypeptide of interest is a therapeutic agent.

38. The method of claim 22, wherein said polynucleotide is administered to a brain ventricle of the subject.

39. The method of claim 22, wherein said polynucleotide is administered to a spinal cord of the subject.


SEQUENCE LISTING (23 sequences)

SEQ ID NO: 1 - DNA, 22 bp, Artificial sequence
  Single strand DNA oligonucleotide
gaccgcggcctcatctcctaca

SEQ ID NO: 2 - DNA, 20 bp, Artificial sequence
  Single strand DNA oligonucleotide
ggccgcccctcttcactctg

SEQ ID NO: 3 - DNA, 20 bp, Artificial sequence
  Single strand DNA oligonucleotide
tcagggaatgggggtcgtca

SEQ ID NO: 4 - DNA, 22 bp, Artificial sequence
  Single strand DNA oligonucleotide
ccaaatccaaatcggctgttca

SEQ ID NO: 5 - DNA, 23 bp, Artificial sequence
  Single strand DNA oligonucleotide
caacgcgaccaggagaggagacc

SEQ ID NO: 6 - DNA, 23 bp, Artificial sequence
  Single strand DNA oligonucleotide
agaggcagagacggggaggaagg

SEQ ID NO: 7 - DNA, 23 bp, Artificial sequence
  Single strand DNA oligonucleotide
aaagaagccgaggcagttgttga

SEQ ID NO: 8 - DNA, 24 bp, Artificial sequence
  Single strand DNA oligonucleotide
cacgcgttaaagggaaaggaatct

SEQ ID NO: 9 - DNA, 20 bp, Artificial sequence
  Single strand DNA oligonucleotide
cgccatcagcagaatctaca

SEQ ID NO: 10 - DNA, 20 bp, Artificial sequence
  Single strand DNA oligonucleotide
cagattggcagaaagcacaa

SEQ ID NO: 11 - DNA, 20 bp, Artificial sequence
  Single strand DNA oligonucleotide
gcttcccttcgactcttcct

SEQ ID NO: 12 - DNA, 20 bp, Artificial sequence
  Single strand DNA oligonucleotide
tctctcaattctgggggttg

SEQ ID NO: 13 - DNA, 20 bp, Artificial sequence
  Single strand DNA oligonucleotide
ctggaacctcatcaccacct

SEQ ID NO: 14 - DNA, 20 bp, Artificial sequence
  Single strand DNA oligonucleotide
ggggccctggtagatgtagt

SEQ ID NO: 15 - DNA, 23 bp, Artificial sequence
  Single strand DNA oligonucleotide
tgcggtgtggagctcgtgcttgt

SEQ ID NO: 16 - DNA, 22 bp, Artificial sequence
  Single strand DNA oligonucleotide
gctaccaggcctttgagatgga

SEQ ID NO: 17 - DNA, 17 bp, Artificial sequence
  Single strand DNA oligonucleotide
gctggcctctttgctaa

SEQ ID NO: 18 - DNA, 22 bp, Artificial sequence
  Single strand DNA oligonucleotide
agtgcatctacatcttcttctg

SEQ ID NO: 19 - DNA, 3196 bp, Mus musculus
caaaaaaatcaataaacagttaaaaaataacattatacaggttatgcttacatatttaaggatgtatatgtaatatatatatgacaacaattaatgggaaaaaggctgtgattctgaaggagagcaagatgaggtataaggtagggtttggagggaggacagagaaaggagaaatgatgtaattgtattataacctcaaaaagatgaggagagagagagagagagagagagagagagagagagagagagagagagagagagatgatgcaggacagccaacagaaaaagcacaggcaaagctgcagcctgatcccttagggagctctggagaatcaatgatacctccagacaaaggggctcttgtcagtcagtgatgctagggctgttcctgaagggaaaactgctggatgcttgggcattcgatctggttttaaggaaaaggaaatctagttttttgaatgcagttcttcaaatagggctccaggttggatgtggacatatatttggaaatgcaggaaagccaagacagcagcacttctaaggactaagtgtgtatgcccctctcccacattcacgattggccctactccaaacggctttgaaacctttgggaggttttcaggttagttgaagctgtgcagatgtggccccaagaaagactctttctcttagaagaagagcagaccctgagacgacacaggaaggccgccatctacaaatcaagaattcctccactgagactgatagcaactgcaaccacacctcagattccccagccctcagaaccaccctacatgtggcattgtgtcatggtgatcagacaacccaggcaaacctgccaggatggaaagtgtgttgagtgtcaacatcaacctgtctttcactctctgggccacaccatccttacctcagctggactctggctatgctctacccatcctgtcctggcccagctcctgcctactgctcaccttgaatcctccaactcactggaggtcttccctttcccattctcctaagcccttcaaaaggtgcctgcaacacctgtcccagaaagtcccaagctcaccacaagaactgcccagagctcctacaatcccctcccttcatctcttttcttttctcgctcaccttttctctacccactgctcccctcctttccccttccctccccttccgttcctctttcccttccttggagactgtggtgtttgtcctgaagatgtcctctttgaccttggcacccactgtgagacaggcaccaggtgtgctccacaaagaggactcaacaaagctgcacaccctcctccgctggtccagtcagctctctccgagcctacaattccaaatactgttgagccctttcctcctgcaagagccccctctgatcgggaccatttgcctgagtctagtaacgtcacttcctgcctaggacttgggaaagaagttccctccctaacccctgtcatctccaaaatgaacaggaagtctgggccattgttcctcagagtttcccttgaagatttcccttgtaccaagtgacttagctaggggtttatggctgtgaagagataccatgaccaaggcaactctcataaaggaaaacattgaattggggctggcttacagtttcagagacttagcttattatcatcatggcgggaagcacagcagggtgcaggcagacttggtgctggagaagctgagagttctacatctcgatctgaaggtagccaggaggcggctggatttgtttcacacttggtgtagcttgagcattttagacctcaaaacttctcgtccacagtgatacatttcctctaacaaagccacacttcctaacagtgccactccccataggccaaaactcatgaatctatgggggccaagcctattcgcaccacaccaaccatggagccaagttctgtgccaaaaaggagggaaaagtgaagaggagacaggaggggaaggccacactcatccaggcagagaaaaatcagagaacagttcctatttatgagccttggcaggtcccactgaggctctgagattggagtgagagagagagagaaagagagagagagagagagagagagagagagagagagagagagagagagagaaagagagagagagagagaaagagcaggatcagggaagagagttgcccaaggagatggagaagaggccctggaaagccagtcattaattattttaggcagaacaaccaaaggaaacagagtttctgagttctccatagcctctattgcatggctgtatctccaggaaaagcctgaccctcttaatcacattcctagaatgccttcccctcaaagccaggggacaaccactgcttaagcttttaacccctgcacaataggtccacttgtcaggagcaaagcctgtgtgctcccaagccctgcacctgtcacctttcagagcagggatgcgcagccccacctggttctgggactcaggatctgcatcccagcactggagtccaaatcccttggagtttccctcccccacatgctcttactgttttggtttgtgttgttgttgttttgatataatttcaaacatacagaagagttgtgcaagactggatcacactcacattccccactgtccaggcctccccatgtgagctacaccaacatgcagatttccacgctcctctaggcttgtgggccagtttgaagagtaacttgggtaactgggattgctcaaacaagtggggagtcctggtcccagtagtctcagccaacccccatgaatcctcgggccctcctggctctgaggagctacagccccagcaatactggtgggaagaccatacacactaagccctgctctgggaactggagtttgtcccctggccgcctgcagaagttgctgcccagagccagctataaataacctggaggcggccgactgtggggtggtgtcactgctaccctgggggaggcgggtgaagaatgccctggtgcccagggctttaccttggtgggtaggtcgggcagggtaggacaggcctaagagagaggccggacagacctcctttggaagcagccacttctggtccccatccctggagcgatcgagcggcaggatctgctgtcccatggggaccccaggctctcttcccagtgcacagcttctcctctgcctgttttccctgcttccagtgctccag

SEQ ID NO: 20 - DNA, 3445 bp, Mus musculus
atgggaaacagggaccatcagggagctacaactgtggtaagggagagctaacatacacagatgtaaaattcaaacagaaggaagttatgcgccaagtttagataatcattagaccctggatctaaacaatccagattccagaggcacaagatggaggaaagagagagagtgatttgaaagggggaatcaagaaaggcttctagaagctcatagtctggtgacgggaaaaggttcctaaaaggaactgttagcagtaaatttagatagacacatttattatctatgaacacatatattaaaacatattttcattacaggtattcacgcagacatacacagtttatacagtattttattttaaatcaagaaaaacaaagcatgtttgatatctcttacaaaccttacaagaccagcgggtctcaactggtctctagaccagtgagtgtcaacttgggagtctcaacccctttagggttatctatatttagatatttacattaccattaataatagtagcaaaattagagtgttgatgtagcaacaaaacatgagtgtcacaatatgaggaactgtattaaaagggttacagcattaggaaggctgagaaccactgttcttgatgaaatgctgaatgtggtccataccccctccttaaagcaacagccaacgtgatgctgccctgttcctgtatggttacgaagtaacatctcagtttctagaactttctgctatgggacagtcacagacacagtgagaactcaagacccaggaagccctacagtttcaaatgttgccacgtgctccaggcctgagggaggagcagcagtcactggacctgtctggcgagaagatgtcagggtgagagtagcttaattccagcttaaattctggtgaagctgtcaagctcggactccatcgtcctctgagatctatgcagttctaagggagggctccatgtcccggtgcttgcatagactcagcgctcggcacactctggggtctacagtgttgggaatttctaaagccggctccactctgtgatttttttcttttaaatgacttattttcaacactcatcggaatgacttctttggaagaaagctgagctggcccacagaataggattgtttcatccatgtttgtctctagattcatggtcttatttgtgacctaccctttcctggtgattttatattttaatattctcaaccatcgtgaagtacaaatctgacgccaaacttaaagcgcatgctaagtacgtcttagaagagtcaaacacatttacctccgtgatagttatcaacattttcatgtagtctatgatacgccagtctatggctatatcaacagcaacatggcaaataaaagctttcaaattattatggtaagattttatttttatggtgagaacaggtaatgatgacaggtattgattttatgggagggggaggggtaatcatcttcatataaattctacagaaaggtgaccaacctcactataactgaaaatatgagtttgtgacctatagttagttgtaaattttaagtgacttaataatggctatgatttcttacttttcttagccactgtaggcacagacttgtgctgttcaacacatatgtaatagcatttaaagctgaatttaataaaagtgaaattgaataaaaaccccacttcctacagaattgtagataaattttaagctctagatagccacccttaactaacttggatactataggtctggatccctcttttccttgtcacatgccaacttccagagccagaataattctctcctcattgatgtattagtagatgctatatgcaccactgtgtttagatattagatagttaaatctcaagaagtatcaagtgatgcacaattcgcacttatctaagtgtcttggtagcttatttactatggagaacgccaaatacatatatctgggacatgatcttggaaattttttttcggaggtctttttagatccaagttaaaaaatgcctgccatggaccctgcttctcttccagttctctcgatgtaagatcaagcagctggaataaccactcctgtctctgtgacagcctaagggtggctgtgttgggagggacggataggctgattgggcacaggtgtgtacacaggtgccacgccccacctgttgctgagtggaaccagtataggaagcaaggccagcaggggatggacaggccactggctgtgtccacgggctgtgatcacgtgctccttacctgctaagttacagggttgaactccaaagtacgcgaggtttctgtataccaagttgctccttcctggatgttaaggaagtctgtttctcaaaggagaataaaactcattttgttggcttgtatagtctctataaacttaaacatagggtaggagaaccctggtcttgcaaatgttagggtggggtctgcggtctttggcatgaataataaaattcagcttgggtttcttggctgcttcgtcactgttgctccaactggttaacatctgatctacctgtggactcagaaatttcggttgaatggtccccatgcatcctcagcctgcccacagctgcttcaggccaggggcagcttccgagttcagtttattgggaagaaacctgtttctggctctggcggataatgaaactttctaccatggcctttctttggaaagattggtttcaggctctttcccctcgtcaggttggagaaaggaacttcaccccctccagccagggctcttaggggcgtgcagggcggctttgtctgccaggcggcggcgccaggaaacgtggagcagagccgcagggctgctggagttagactagtctagaactttcttccgctgggtccaggagcagcggagttccggccaggacctgggggcgtgcccgggtcgaggtcgggtcggctgcgggcctgagagcgcgcggggctggggtgggaggagcggtcgcgcggggaggagccggcgggaggaggcggcgctcccgcgggccgagcggcgagctgggacccggcggccctgaccccgcggctccgcggagggcgagcgcggccgcggacaaaggagcatgtcgtcgcgaagcgggaccgatcctcgggccgccgggaggctcgggccaccgccagggccgccccgccccgccgccgcccgctaggccggtgccgtctctcggccccgcaggcccggtcagcggcatggagccgccgccgccgctgctgctgctgccgctggcgctgctcgctctgctgtggggaggagagcgcggggccgcggcgctgcccgcgggctgcaagcacgacggtcgggcccggggtaccggccgagcggccgccgccgccgaggggaaggtggtgtgcagcagcctggagctcgcgcaggttctgcccccggacacgctgcccaaccgcacggtcaccct

SEQ ID NO: 21 - DNA, 3146 bp, Mus musculus
gaagaaagacacactggacaagacaggttcaagcacgtccagtacaaaacaaaaacaaaaaacaacaacaacaaaaaagaaaacatgtatattcaacaatgaagtaagcaatcttagccaggctctcctttctagagaacattctcacagaacactcaaaaccctcttgctccaatcccttgattatcagaagaccatctgactgttaattaaatcagcattatggttttaaaatgagtctttatgatttgcttagcattaatcaaacctccaggtcttatccacctcaaggggagctaacaaaattgaattctttgacctgcaaagattcagagccccaaacactgctatttctcttctcctaactcccttaccaggaggcttagtgcaagcatttggcccacctagtccccttcctgctaatcagcttactgcactagcattagcgagcattccagggcctacaaactgctggctaggtgtttgtttggaccttcagaaaacaaatgaagagattgtttaggagatgaaaagatgtattgaacgaggttgtacattttaagggagaaggccgagaagactctctaatcaagagccagccagggttgttcattaactaccataaatttggtagtagggagagagatgtggtcagtaactcaatctctcattcacctaaatgaaacatgtaagctatggcaccgcagccaggatgtgaagaaaaccagtaaagggctaagcaaagacacctccttctaacttaaacactacctccaacacccttatgttctctaggtagttgctgttagtattaccccagaacaggcaatgtcttcagcagaagccccacaaaggcggtgaattttgacacaagtccatccctcatcatggattcctgtaaccatcctctgagaagagtttttacaatttcaactcaatatgtgaaaccacaccttcctttctttagaaaagtatgattgattatccatgggacctattcacagcacaaagtgactcaaaggcaaaaagcacttgggcttctctggggggcaactgccctttatctcacacccatgtcttggacaacaaatgttttcttactctgtctgttttttctgtcctgctctgtcacataaaatcctgcccgacctttgactcaaactccagacagtctacctgctgatcgcccggcccctgttcaaacatgtcctaatactctgtctctgcaagggtcatcagtagttttccatcttactcaacatcctcccagtgcttcaaagcacctcactttatcttcacatccttgtctctttctaattaaagtttaaaaagttggtttctaaggctgatggaggtctaaaaaacaagcaaatcaaagacctgagggtgttctaatttacttggtagttggtcagaactaccagtatgttatggtcagagataaattagggatactatacttagatcaaaatttataaaaagacagagtagagaggatctctgtgagttcaaggttagcctggtcttcgtgcagcgagctccaggctagccaaaggtacaaaatgagattctgtccctaaacagcaaaccaaaaactaaataaaattgataaatgagcatagttcattatgatgaatgctctttacttgtgttagacacagggttgggtgaggcatcacagtgataacagttacagatgcacttagggtatgacactgcctgcagagcacatttgtcaaggaagactaaagccttctggccatgtcctcagggtttacatgaccttatctgaaatgtgtctccagttcaattatctggaatgtgtgtctgcttcagtgcctcacattggtagagaaacattgaccagtgggaggaagccagacaacaaagtcctgggaagagaggattccaggtcctgtgtctgacgaggaatctctcaaagaggtgggcatgcttatttagcaaaagaaaatatgctgtcagaagaccagtaatcataacaaatttataaagaggctgcgctttgtcatggatcgagaatatgggatgtacattaaaaacacacagacaatcagacgtaccagtagtcatgtaatctggcttcagagtgggagagaagtcaggaaaccgagatgtcccaacatgggaccccatagattattttcatgtgaatgtaaggcaactgctgtcatcttccagtttctcaagacaagccaaagtccaggttttaatgcaacgtggttcaatttgcaatttttgcagatatttcaaaatcctagagaaatggtagagcctgtggtcaggtggcagtccagctttgccagtttacgagatcctgggaggcaattcttagtttcaatggattgtggagttcagtagtgtggagttgacatgtgtgggtgagagattttactggatagtgattcctgtatgaagagggctttccatcacattctgagcaatggaaagtaatgatgtcaaccttggttgacaatgcacaagagattctggagaaagcatctccttcttaggcacagatattgaatggaatcatctgacatttgtattttccagtttataaaatgcctttatatcttgtcacatttaaagttcttagaaaatctccttcaaagagaaatgcgatatttctgatttacaaatgtgtggtctgatatctgagataggaaatcatttccagaagcaagcaggacattgagtagcagatctgggattgggtgtgtcagagcctccaacactgtcagactcaaaggtgcaggacaataagtagtcttactctggctgtatcttctcattgttgcttttggacggttgccctctttcccaaaggtgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgacaggatggcttcccttcgactcttcctcctttgcctcgctggactggtatttgtgtctgaagctggccccgcg

SEQ ID NO: 22 - DNA, 564 bp, Mus musculus
atgcggctgcggctgctggtgtccgcgggcatgctgctggtggctctgtcgtcctgcctgccttgcagggccctgctcagcaggggatccgtcccccgagcgccgcgggccccgcagcccttgaatttcttgcagccggagcagccccagcaacctcagccggttctgatccgcatgggtgaagaatacttcctccgcctggggaatctcaacagaagtcccgctgctcggctgtcccccaactccacgcccctcaccgcgggtcgcggcagccgcccctcgcacgaccaggctgcggctaactttttccgcgtgttgctgcagcagctgcagatgcctcagcgctcgctcgacagccgcgcggagccggccgaacgcggcgccgaggatgccctcggtggccaccagggggcgctggagagggagaggcggtcggaggagccgcccatctctctggatctcaccttccaccttctgcgggaagtcttggaaatggcccgggcagagcagttagctcagcaagctcacagcaacaggaaactgatggagattatcgggaaatga

SEQ ID NO: 23 - DNA, 411 bp, Mus musculus
tgaagccagtggtccaggagctggcctctttgctaagcattcttcactctgtgtcttgatgtcccttagagtggacatgatcctcaaactgatggccggcattctactgctgactgtgtgtttggaaggctgctccagccagcactggtcctatgggttgcgccctgggggaaagagaaacactgaacacttggttgagtctttccaagagatgggcaaggaggtggatcaaatggcagaaccccagcacttcgaatgtactgtccactggccccgttcacccctcagggatctgcgaggagctctggaaagtctgattgaagaggaagccaggcagaagaagatgtagatgcactggcccaggtggatccacaacacccgagtataacattgaccctgaggcttgtgacc